(19) United States
(12) Patent Application Publication
(10) Pub. No.: US 2010/0215708 A1
(43) Pub. Date: Aug. 26, 2010

(54) COATING OF DEVICES WITH EFFECTOR COMPOUNDS

(76) Inventors: Andreas Zumbuehl, Nyon (CH); Danial S. Kohane, Newton, MA (US); Lino Da Silva Ferreira, Coimbra (PT); Robert S. Langer, Newton, MA (US); Gerald R. Fink, Chestnut Hill, MA (US)

Correspondence Address: Pearl Cohen Zedek Latzer, LLP, 1500 Broadway, 12th Floor, New York, NY, 10036, US

(21) Appl. No.: 12/306,794

(22) PCT Filed: Jun. 28, 2007
(86) PCT No.: PCT/US07/15052

§ 371 Date: Dec. 24, 2009

Related U.S. Application Data
(60) Provisional application No. 60/817,075, filed on Jun. 29, 2006.

(51) Int. Cl. A61F 2/00; A61K 31/7088; A61K 31/713; A61K 31/7048; A61K 9/16; A61K 9/70; A61P 31/00; A61M 25/00; A61M 27/00; A61M 16/04

(52) U.S. Cl. 424/422; 514/44 R; 514/44 A; 514/31; 424/497; 424/443; 604/265; 604/544; 604/540; 128/207.14

(57) ABSTRACT

This invention is directed to substrates, materials and devices coated with a gel, foam, film, particle or composition comprising a polymei solvent and effector compounds attached thereto, processes of producing the same, and methods of use thereof, of in, inter-alia, biological applications, including preventing infection and the treatment of various diseases.

### FIELD OF INVENTION

This invention relates to devices coated with effector compounds, processes of producing the same, and methods of use thereof and their use in biological applications, including preventing infection and the treatment of various diseases.

### BACKGROUND OF THE INVENTION

Intravascular catheter infections are a major cause of morbidity and mortality in hospitalized patients, accounting for the majority of the 200,000 nosocomial bloodstream infections occurring in the USA annually. Fungal sepsis is a leading cause of death in patients with indwelling vascular catheters, particularly in the immunocompromised. In many instances the portal of entry of the fungus is via the skin. For example, Candida is the fourth most common cause of bloodstream infection in hospitalized patients. Up to 40% of patients with Candida isolated from intravenous catheters actually have fungemia, and the mortality rate of patients with catheter-related candidemia approaches 40%. Obviously there is great and immediate need for novel materials, that could mitigate biofilm formation on medical devices

Many microbes in their natural habitats are found in a protected microenvironment, attached to a surface, and not as free-floating organisms. Biofilms are embedded within a matrix of extracellular polymers, and characteristically display a phenotype that is markedly different from that of individual cells. Most important, they are significantly less susceptible to antimicrobial agents.

The coating of surfaces with an effector compound, such as an antifungal, has numerous applications, including, sterilization and/or controlled delivery of the compound. To date there are no methods of biofilm mitigation, or surface/local release systems that kill or prevent Candidal infection in vivo. Current techniques include the application of compounds to, for example, a catheter surface or impregnation of the catheter, yet there is no technique known to date, which addresses these limitations.

### SUMMARY OF THE INVENTION

In one embodiment, this invention provides a coated material comprising:
a. a substrate;
b. a gel or film comprising a polysaccharide attached on at least a portion of a surface of said substrate; and
c. at least one effector compound associated with said gel or film.

According to this aspect, and in one embodiment, the polymer is a block copolymer. In another embodiment, the polymer is a polysaccharide, which in some embodiments is a poly(pyranose) or a poly(furanose) or a combination thereof, or in other embodiments, is a dextran or an inulin.

In one embodiment, the compound is covalently associated with the gel or film, or in another embodiment, the compound forms a physical interaction with the gel or film

In one embodiment, the substrate is a part of, or in the form of a bead, microparticle, nanoparticle, bandage, suture, catheter, stent, valve, pacemaker, conduit, cannula, appliance, scaffold, central line, pessary, tube, drain, trochar or plug. In one embodiment, the catheter is a PA, pericardial, pleural, urinary or intra-abdominal catheter.

In one embodiment, the drain is a cerebrospinal fluid drain.

In one embodiment, the tube is a tracheostomy, endotracheal or chest tube.

In another embodiment, the substrate is a part of, or in the form of an implant, a rod, a screw, or an orthopedic appliance. In another embodiment, the substrate is a part of, or in the form of a pipe lining, a reactor, or equipment which comes into contact with food or seawater or wastewater.

In one embodiment, the compound is realeased slowly, over a course of time, or in another embodiment, the compound is minimally released over a course of time.

In one embodiment, the coated material may be affixed, glued, or sutured to the skin, or pierce the skin, or in another embodiment, the coated material serves as a portal through which other coated material s are passed through the skin.

In another embodiment, the effector compound is an antibiotic, an antiviral, an antifungal, an anti-helminth, an anti-inflammatory, an antihistamine, an immunomodulatory, an anticoagulant, a surfactant, a bronchodilator, an antibody, a beta-adrenergic receptor inhibitor, a calcium channel blocker, an ace inhibitor, an ABC Transporter Inhibitor, a Multi-drug resistance transporter inhibitor, a growth factor, a hormone, a DNA, an siRNA, an shRNA, an mRNA, an miRNA, an smRNA, an agRNA a vector or any combination thereof. In one embodiment, the compound is a polyene antifungal, which in another embodiment is Amphotericin B.

In another embodiment, this invention provides a process for preparing a coated material comprising a gel or film covalently attached thereto comprising a polymer and an effector compound, said process comprising the steps of:
a. preparing a gel or film comprising a polymer;
b. chemically reacting said gel or film with said effector compound; and
c. attaching said gel or film in (b) to at least a portion of a surface of a substrate, thereby preparing a coated material.

According to this aspect of the invention and in one embodiments, preparing the gel or film comprises the step of dispersing the polymer in water, dimethylsulfoxide, dimethylformamide or N-methylpyrrolidinone (NMP).

In one embodiment, reacting comprises activating the gel to produce an active ester, amine- or thiol-reactive group or photoreactive group in the gel. In one embodiment, the ester is N-hydroxy-succinimide ester.

In one embodiment, attaching the gel to the coated material is via chemically reacting said gel with at least a portion of a surface of said coated material. In another embodiment, attaching comprises activating at least a portion of a surface of the coated material, and providing conditions whereby the gel reacts with the activated surface of the coated material

In another embodiment, this invention provides a process for preparing a coated material comprising comprising an effector compound associated thereto, said process comprising the steps of:
a. preparing a gel or film comprising a polymer dispersed in a solvent;
b. loading said gel or film with said compound; and
c. attaching said gel or film in (b) to at least a portion of a surface of a substrate; thereby preparing a coated material.

In another embodiment, this invention provides a preventing, diminishing or reducing the incidence of infection caused by introduction or implantation of a substrate in a subject, the method comprising attaching to a portion of a surface of said substrate, a gel or film comprising a polysaccharide and at least one effector compound, wherein said effector compound is associated with the prevention, diminishment or reduction in incidence of infection.

In another embodiment, the invention provides a method of preventing, diminishing or reducing the incidence of local or systemic fungal infection in a subject, said method comprising contacting a site of, or predisposed to infection with:
a. a substrate
b. a gel or film comprising a polymer attached to said substrate in (a), on at least a portion of a surface of said substrate; and
c. said gel or film being associated with at least one effector compound;
   wherein said at least one effector compound is associated with the prevention, diminution or reduction of the incidence of said fungal infection. In one embodiment, the gel or film association with the at least one effector compound increases the solubility of the compound, or in another embodiment, provides a better formulation for the compound, enhancing its activity

In one embodiment, the method results in diminished systemic toxicity of the effector compound. In one embodiment, the substrate is a bead or particle ranging in size from about 20 nm-3000 micron. In another embodiment, the substrate is a contraceptive device.

In another embodiment, this invention provides a method of preventing, diminishing or reducing the incidence of microbial attachment to a biomedical substrate, the method comprising attaching to a portion of a surface of a substrate, a gel or film comprising a polymer comprising at least one effector compound, wherein said at least one effector compound is associated with the prevention, diminishment or reduction in incidence of microbial attachment to said substrate.

In one embodiment, the invention provides a method of controlled release of an effector compound in a subject, said method comprising administering to or implanting in said subject:
a. a substrate
b. a gel or film comprising a polysaccharide attached thereto, on at least a portion of a surface of said substrate; and
c. at least one effector compound associated with said gel or film;
   whereby said effector compound is released slowly, as a function of time, from said gel or film

In one embodiment, the invention provides a topical composition for controlled delivery of a compound of interest, the composition comprising:
a. a particle
b. a gel or film comprising a polymer attached on at least a portion of a surface of said particle; and
c. at least one compound of interest associated with said gel or film.

In another embodiment, this invention provides a method of topical controlled delivery of a compound of interest to a subject, said method comprising topically administering to said subject a composition comprising:
a. a particle
b. a gel or film comprising a polymer attached on at least a portion of a surface of said particle; and
c. at least one compound of interest associated with said gel or film.

In another embodiment, the invention provides a method of controlled delivery of a compound of interest to a subject, said method comprising administering to said subject a composition comprising:
a. a particle
b. gel or film comprising a polymer attached on at least a portion of a surface of said particle; and
c. at least one compound of interest associated with said gel or film.

In another embodiment, the invention provides a method of treating, preventing, diminishing incidence, prolonging remission, prolonging latency, preventing relapse, preventing latency, ameliorating symptoms, or a combination thereof, of a disease in a subject, said method comprising administering to said subject:
a. a substrate
b. gel or film comprising a polymer attached on at least a portion of a surface of said particle; and
c. an effector compound associated with said gel or film;
   whereby said effector compound is associated with treating, diminishing incidence, prolonging remission, preventing relapse, ameliorating symptoms of a disease in said subject.

In another embodiment, the invention provides a method of treating, preventing, diminishing incidence, prolonging remission, prolonging latency, preventing relapse, preventing latency, ameliorating symptoms, or a combination thereof, of a disease in a subject, said method comprising administering to said subject:
a. a particle
b. gel or film comprising a polymer attached on at least a portion of a surface of said particle; and
c. a compound of interest associated with said gel or film;
   whereby said compound of interest is associated with treating, diminishing incidence, prolonging remission, preventing relapse, ameliorating symptoms of a disease in said subject.

In one embodiment, the invention provides a method of retaining an effector compound in a subject in active form, said method comprising administering to or implanting in said subject:
a. a substrate
b. a gel or film comprising a polymer attached on at least a portion of a surface of said substrate; and
c. at least one effector compound associated with said gel or film;
   whereby said effector compound is associated with said gel or film and retains activity for a prolonged period of time. In some embodiments said prolonged period of time is at least 10, 20, 30, 40, or 50 days. In some embodiments said prolonged period of time is up to 20, 30, 40, 50, 60, 70, 80, 90, or 100 days. In some embodiments the compound, or a surface having said compound associated therewith, retains at least 25%, at least 50%, or at least 75% of its activity at the end of said time period. In some embodiments said activity is killing a pathogenic microorganism, which in some embodiments is a fungus.

### BRIEF DESCRIPTION OF THE DRAWINGS

FIG. 1 schematically depicts the hydrogel, amphotericin B (AmB), and formation of the loaded hydrogel via dextran prepolymer reaction with Acryl-PEG-NHS to create a hydrogel, followed by surface modification to incorporate AmB (A-C). FIG. 1D is a schematic representation of the preparation of amphogels. FIG. 1E graphically depicts gel characteristics. Swelling ratio for dextran-based hydrogels without AmB and amphogels in PBS for several days, at 25° C. (average±S.D., n=3) (panel 1). Representative mechanical spectra for amphogels showing G′ exhibited a plateau at lower frequencies and G″ was typically 1 order of magnitude smaller than G′, representing a predominantly elastic behaviour (panel 2). Loading efficiency of amphogel was determined (panel 3). AmB dose response in terms of fungal viability was determined (panel 4). Candida was exposed to Amphogels loaded with different concentrations of AmB or culture media comprising the same and fungal survival was assessed by colony growth assay.

FIG. 2 schematically depicts establishment of fungal biofilm formation and survival protocols. In one type of experiment that studied the effect of direct exposure to the discs (FIG. 2B), the discs were placed in media containing fungi, and the extent of fungal survival in the media (FIG. 2B-1) and on the disks surfaces (FIG. 2B-2) was determined. In a second type of experiment designed to determine the effect of AmB released from the discs, they were first incubated in media without fungi. After that incubation, the media were removed, fungi were added to that incubation medium, now without the disc, and survival was assessed (FIG. 2C-1). In both contexts, discs were used repeatedly in serial experiments describing the time course of fungicidal activity.

FIGS. 3A and B plots the results of XTT viability assays of the yeast cultured on polyurethane; dextran hydrogels with different pore sizes; dextran hydrogels with different amounts of PEG incorporated into the gel; and those loaded with amphotericin B. FIG. 3C demonstrates Candida albicans viability on the hydrogel surface as assessed by a colony growth assay after a 2-hour exposure to dextran-based hydrogels with or without AmB. FIG. 3D are SEM images of dextran-based gels without (panel 1) and with (panel 2) AmB incubated with C. albicans for 48 h. FIG. 3E-F demonstrates yeast killing by amphogels as assessed by a colony growth assay, as well. (E) plots yeast cell killing in media upon contact with amphogel as a function of time. Over 99% of Candida albicans cells exposed to amphogel were killed after 120 min. (F) plots survival of fungi added to media that had been exposed to amphogels as a function of time. The individual media samples had been exposed to the gels for a time period equal to the interval between the predetermined time points. ** denotes statistical significance (P<0.001, n≧5) between the various samples and cells cultured without amphogels.

FIG. 4 plots the viable colonies obtained. PEG=poly(ethylene glycol). 5000=MW of the PEG acrylate added. 40 mg amount of PEG acrylate added to the reaction. The active esters were reacted with amphotericin B or hydrolysed in the presence of water

FIG. 5 shows SEM micrographs of Candida albicans on polyurethane) disks (A-C; G, H), and dextran hydrogels (D) without amphotericin B. Note the biofilm Candida albicans blastosphores on Amphogels (E-F; I-J). Note absence of biofilm.

FIG. 6A demonstrates that repeat washing of gels does not diminish antifungal activity. FIG. 6B demonstrates the effect of culture well diameter and mechanical agitation on the killing of Candida albicans. Fungal survival on the surface of the gel disk or in the surrounding media (average±S.D., n=4). “Control”: cells not exposed to amphogels.

FIG. 7 plots results of an XTT assay of Candida on amphotericin B-adherent, poly(urethane)-poly(lactic co-glycolic)acid polymer matrices.

FIG. 8 plots the retention of Amphotericin B in different sugar-based polymeric matrices.

FIG. 9 plots a time-course experiment of Candida exposed to Amphogel surface for a set time.

FIG. 10 demonstrates the biocompatibility of hydrogels. 10A. Hemolysis assay for amphogels. No release of hemoglobin was observed after exposure to amphogels Red blood cells were suspended in PBS 7.2 at 2×10⁸ cells/0.9 mL. The following treatments were added: 100 μL of double distilled water (“H₂O”), 100 μL PBS (“untreated”), or 100 μL PBS plus one amphogel disk. After 1 h incubation (37° C.) the cells were centrifuged (4,000 rpm, 10 min., 4° C.) and the free haemoglobin in the supernatant was quantified by optical density measurement at 540 nm (average±S.D., n=4). The untreated and H₂O groups were negative and positive controls, respectively. 10B-10G demonstrate the in vivo biocompatibility studies and biological activity of amphogels. B,C) Representative light micrographs of amphogel implanted subcutaneously and surrounding tissue after 3 days (B) or 3 weeks (C), stained with hematoxylin-eosin. Minimal to mild inflammation was observed at day 3 and only mild to moderate inflammation at 3 weeks. In B and C: original magnification ×50: C: ×200. D, E, F, G) SEM photographs from the surface of amphogel (D, E), or dextran gel without AmB (F, G) incubated with C. albicans that were implanted into mice and then removed after 5 days. Amphogels did not have any Candida biofilm (D), and had only a few host cells (mainly white blood cells) (E). Dextran gels without AmB were covered with fungal biofilm (F, arrow). Certain areas of the disks were covered with a large number of Candida cells (G, arrow) mixed with white blood cells (arrow head).

### DETAILED DESCRIPTION OF THE INVENTION

The invention is directed to, in some embodiments, coated materials, and their use in, inter-alia, biological applications and devices and/or materials for use in such applications. In some embodiments, such materials find use in the preparation of surgical devices and instruments, upon which, for example formation of biofilms does not occur, or the surface is fungicidal or bactericidal, or controlled release and delivery of compounds of interest can be accomplished

In some embodiments, this invention provides a versatile platform for creating substrates, particles, rods, spheres, gels, foams, films, compositions, devices, kits, etc., on or within which, compounds of interest are associated, and can be released in a controlled manner, as a function of time, or in another embodiment, their release is minimal to none.

As exemplified herein, and representing one embodiment of the invention, the invention provides a versatile platform, for example, use of a gel, to which a compound of interest is bonded, or in some embodiments, physically associated, where, even under circumstances where the physical association is non-covalent, nonetheless, leakage of the compound of interest from the gel was minimal to none, indicating the versatility and longevity of the activity and application of compositions and materials of this invention.

In one embodiment, this invention provides a coated material comprising:
a. a substrate;
b. a gel or film comprising a polymer attached thereto, on at least a portion of a surface of said substrate; and
c. at least one effector compound associated with said gel or film.

In another embodiment, this invention provides a coated material comprising:
a. a substrate;
b. a foam or particles comprising a polymer attached thereto, on at least a portion of a surface of said substrate; and
c. at least one effector compound associated with said foam or particles.

In one embodiment the term “coated” refers to the physical attachment, or, in another embodiment, association of a gel, film, foam, particle and/or composition of this invention with at least a portion of a surface of a material whose “coating” is desired. In one embodiment, such coating will comprise less than 1% of an exposed surface of the material, or in another embodiment, from 1-10%, or in another embodiment, from 1-25%, or in another embodiment, from 1-50%, or in another embodiment, from 1-75%, or in, another embodiment, from 1-100% of at least one surface of the material.

In one embodiment, application of such “coating” will be in a pattern, or on specific regions of the material to suit a particular purpose. For example, and in some embodiments, tubing may comprise coating of one material on the luminally exposed surface of the tube, for example, a coating comprising an anti-inflammatory compound, which, in some embodiments, may be coated with a different material, for example, a coating comprising a compound to prevent biofilm formation.

The coating applied to the materials of this invention may comprise gels, foams, films, particles and compositions comprising a single, or, in other embodiments, multiple effector compounds and/or other compounds of interest. In one embodiment, when multiple compounds are applied, they may be applied as part of a single composition, gel, foam, film or particle type, or in other embodiment, in a series of the same or different compositions, gels, foams, films or particles. In one embodiment, when multiple compounds are applied, each may be applied in an individual composition, gel, foam, film or particles.

For example, a material for application in a subject with a cardiovascular disease or condition may be administered a coated material, such as a stent or baloon catheter, which may comprise the same types of compounds, for example, one or more antibacterial compounds, or different types of compounds, for example, statins, antiinflammatory compounds and antibacterial compounds, and multiple compounds of each type. Such compounds may each be within a single gel, coating the stent, or in multiple gels coating the stent, or in some embodiments, some compounds may be applied in a gel formulation to the stent, while others are applied as particle formulations to the stent.

In one embodiment, the coating of a material will be on at least one surface of the material, or in another embodiment, on two or more surfaces of the material, of in another embodiment, on every exposed surface of the material, or in another embodiment, on any surface of the material.

In one embodiment, the term “coated material” applies not only to a surface coating of the material, but is to be understood as encompassing embedding and/or impregnating the material, in whole, or in some embodiments, in part, with the gels, films, foams, particles and/or compositions described herein. In some embodiments, the embedding and/or impregnating the material may be according to a desired pattern and/or design, to suit a particular purpose or application. In some embodiments, multiple coatings may be impregnated or embedded in the material, each of which may be applied according to a particular pattern or design, which may be the same, or in another embodiment, different than the patterning of a first coating.

In some embodiments, the embedding and/or impregnating of the material may be to a particular surface of a material, in a particular pattern and/or design, to suit a particular purpose or application. In some embodiments, the embedding and/or impregnating of the material may be to two or more surfaces of the material in the particular pattern and/or design, or such pattern and/or design may vary as a function of the surface to which the material is being embedded and/or impregnated within.

According to these aspects, and in one embodiment, the gel, film, kits or compositions of this invention comprise a block copolymer, or in another embodiment, the gel, film, kits or compositions of this invention comprise a protein-based polymer, or in another embodiment, the gel, film, kits or compositions of this invention comprise a sugar-based polymer, or in another embodiment, the gel, foam, film, kits or compositions of this invention comprise a polymer blend, or in another embodiment, the gel, foam, film, kits or compositions of this invention comprise any polymer which is suitable for the particular application, as will be appreciated by one skilled in the art.

In some embodiments, the term “gel” encompasses its ordinary meaning in the art. In one embodiment, the term “gel” refers to a composition comprising a polymer having a fluidity at room temperature between that of a liquid and a solid. In some embodiments, the term “gel” refers to a solid or semisolid colloid system formed of a solid continuous phase and a liquid phase (either discontinuous or continuous or mixed), which, in some embodiments, can be identified by its outward gelatinous appearance, and/or exhibits properties of a solid such as plasticity, elasticity, or rigidity. In some embodiments, the liquid phase can be a ‘dispersed’ phase, or in other embodiments, continuous. In some embodiments, the gelling component (solid phase) is lipophilic and present in concentrations of less than 10, or in another embodiment, 15, or in another embodiment 20, or in another embodiment, 25, or in another embodiment, 30, or in another embodiment, 40 percent. In some embodiments, the term “gel” may encompass a silica gel, an aluminosilicate gel or other materials, which are primarily solid and/or particulate, microspheroidal, spheroidal, etc., or described with descriptive properties, terms, or expressions which indicates destruction of the two-phase system, such as, pore volume, pore diameter, surface area. In one embodiment, the gel is a hydrogel, or in another embodiment, the gel comprises polymers dispersed in solvents other than water or aqueous solutions.

In some embodiments, this invention provides a gel comprising a compound as herein described. In some embodiments, the compound is covalently attached to the gel, or in some embodiments, the compound is associated with the gel, by methods as herein described.

In some embodiments, the term “foam” encompasses its ordinary meaning in the art. In some embodiments, the term “foam” refers to a colloidal suspension of a gas in a liquid. In one embodiment, the term “foam” refers to a composition comprising an internal phase of gas in an external phase of a liquid or solid. In a liquid foam, in some embodiments, a colloidal adsorptive agent forms a film that bounds a gas bubble, with the colloidal dimension in the foam affecting the thickness of the film, not the size of the bubble.

In some embodiments, this invention provides a foam comprising a compound as herein described.

In some embodiments, the term “film” encompasses its ordinary meaning in the art. In one embodiment, the term “film” refers to condensed matter restricted in one dimension. In some embodiments, the term “film” refers to a symmetric film, or in other embodiments, an assymetric or in other embodiments, a thin film, or in other embodiments, a thick film, or in other embodiments, an open film, or in other embodiments, a closed film, or in other embodiments, a partly open film, or in other embodiments, a stable film, or in other embodiments, a metastable film, or in other embodiments, a stratified film, or in other embodiments, any film which is applicable, as will be appreciated by one skilled in the art.

In some embodiments, this invention provides a film comprising a compound as herein described. In some embodiments, the compound is covalently attached to the film, or in some embodiments, the compound is associated with the film, by methods as herein described

In one embodiment, the gel, foam, film, kits or composition comprises a polymer, which in some embodiments is a polysaccharide. In some embodiments, the polymer is a homopolymer, or in some embodiments, the polymer is a block co-polymer. In some embodiments, such polymers will comprise ester or amide linkages. In some embodiments, such polymers will, when used to prepare a gel, film, particle, etc. or any coated material of this invention, will impart properties to the material such that, in some embodiments, erosion of the substrate is minimized, or in some embodiments, comparable elastiticity, flexibility resistance to stress or strain, or other physical characteristics of the substrate are maintained, or in some embodiments, enhanced, to suit the particular application of use of the coated material. In some embodiments, the polymer will have free hydroxyl groups.

In some embodiments, the polymer is a polysaccharide. In some embodiments, the polysaccharide is a poly(pyranose) or a poly(furanose) or a combination thereof. In some embodiments, the polysaccharide is a dextran, an inulin or a glycosaminoglycan.

In some embodiments, the polymer choice is a reflection of the effector compound, whose incorporation within the coating is desired, so as to maximize retention of the compound within the coating.

In some embodiments, the polymer may comprise, inter-alga, poly (pyranose), poly(hydroxyl acid), poly(lactone), poly (amino acid), poly(anhydride), poly (urethane), poly (orthoester), poly (phosphazine), poly(phosphoester) or poly (lactic-co-glycolic) acid, poly(ether ester)s, synthetic poly(amino acids), polycarbonates, poly(hydroxyalkanoate)s, and poly(ε-caprolactone)s.

In one embodiment, the polymer is a synthetic polymer, or in another embodiment, the polymer is a natural polymer. In one embodiment, the polymer is a poly(cianoacrylate), poly(allyl-cianoacrylate), poly(ketal), poly(caprolactone), poly(acetal), poly(α-hydroxy-ester), poly(β-hydroxy-ester), poly(hydroxyl-alkanoate), poly(propylene-fumarate), poly (imino-carbonate), poly(ester), poly(ethers), poly(carbonates), poly(amide), poly(siloxane), poly(silane), poly(sulfide), poly(imides), poly(urea), poly(amide-enamine), poly(organic acid), poly(electrolytes), polyp-dioxanone), poly(olefin), poloxamer, inorganic or organomatallic polymers, elastomer, or any of their derivatives, or a copolymer obtained by a combination thereof.

In one embodiment, the polymer comprises poly(D,L-lactide-co-glycolide) (PLGA). In another embodiment, the polymer comprises poly(D,L-lactide) (PLA). In another embodiment, the polymer comprises poly(D,L-glycolide) (PGA) or poly(glycerol sebacate), PGSA. In one embodiment, the polymer comprises a glycosaminoglycan.

In one embodiment, the polymer may comprise proteins such as zein, modified zein, casein, gelatin, gluten, serum albumin, collagen, actin, α-fetoprotein, globulin, macroglobulin, cohesin, laminin, fibronectin, fibrinogen, osteocalcin, osteopontin, osteoprotegerin, or others, as will be appreciated by one skilled in the art. In another embodiment, the polymer may comprise cyclic sugars, cyclodextrins, synthetic derivatives of cyclodextrins, glycolipids, glycosaminoglycans, oligosaccharides, polysaccharides such as alginate, carrageenan (χ, λ, μ, κ) chitosane, celluloses, chondroitin sulfate, curdlan, dextrans, elsinan, furcellran, galactomannan, gellan, glycogen, arabic gum, hemicellulose, inulin, karaya gum, levan, pectin, pollulan, pullulane, prophyran, scleroglucan, starch, tragacanth gum, welan, xanthan, xylan, xyloglucan, hyaluronic acid, chitin, poly(3-hydroxyalkanoate)s, such as poly(β-hydroxybutyrate), poly(3-hydroxyoctanoate) or poly(3-hydroxyfatty acids). In another embodiment, the polymer may comprise chemical derivatives thereof (substitutions, additions, and elimination of chemical groups, for example, alkyl, alkylene, hydroxylations, oxidations, and other modifications routinely made by those skilled in the art), blends of, e.g. proteins or carbohydrates alone or in combination with synthetic polymers.

In one embodiment, the polymer comprises synthetically modified natural polymers, and may include cellulose derivatives such as alkyl celluloses, hydroxyalkyl celluloses, cellulose ethers, cellulose esters, nitrocelluloses, and chitosan. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate and cellulose sulfate sodium salt.

In one embodiment, the polymer comprises synthetic degradable polymers, which may include, but are not limited to polyhydroxy acids, such as poly(lactide)s, poly(glycolide)s and copolymers thereof; polyethylene terephthalate); poly(hydroxybutyric acid); poly(hydroxyvaleric acid); poly[lactide-co-(ε-caprolactone)]; poly[glycolide-co(s-caprolactone)]; poly(carbonate)s, poly(pseudo amino acids); poly(amino acids); poly(hydroxyalkanoate)s; poly(anhydrides); poly(ortho ester)s; and blends and copolymers thereof.

In one embodiment, the polymer comprises a bioerodible polymer such as poly(lactide-co-glycolide)s, poly(anhydride)s, and poly(orthoester)s, which have carboxylic groups exposed on the external surface as the smooth surface of the polymer erodes, which may also be used. In one embodiment, the polymer contains labile bonds, such as polyanhydrides and polyesters.

In one embodiment, the polymer is biodegradable. In one embodiment, the term “biodegradable polymer” refers to a material, which is degraded in the biological environment of the cell or subject in which it is found. In one embodiment, the biodegradable polymer undergoes degradation, during which, acidic products, or in another embodiment, basic products are released. In one embodiment, bio-degradation involves the degradation of the polymer into its component subunits, via, for example, digestion, by a biochemical process. In one embodiment, biodegradation may involve cleavage of bonds (whether covalent or otherwise) in the polymer backbone. In another embodiment, biodegradation may involve cleavage of a bond (whether covalent or otherwise) internal to a side-chain or one that connects a side chain to the polymer backbone.

In one embodiment, this invention provides a coated material as described herein, wherein the substrate is a particle, which is of any size which finds application in the methods as described herein, in some embodiments. In some embodiments, the particle is of a diameter ranging from about 1-900 nanometer, or in another embodiment, the particle is of a diameter ranging from about 1-900 micrometer, or in another embodiment, the particle is of a diameter ranging from about 1-10 millimeter.

In one embodiment, the particle comprises any polymer of this invention, and can be prepared by methods well known in the art. In another embodiment, the particle comprises a core to which a polymer as described herein is attached. According to this aspect, and in one embodiment, the core is a metal which is functionalized, such that a polymer can be attached thereto, by conventional means. In some embodiments, the particle is coated with a gel, film or foam of this invention. In some embodiments, the coating of the particle is via formation of a bond between elements of the coating and the particle core, which in some embodiments, is a covalent bond. In some embodiments, the gel, film, or foam is wrapped around the particle.

In another embodiment, the gels, films, foams, particles, and/or compositions of this invention may comprise sugars, cyclic sugars, cyclodextrins, synthetic derivatives of cyclodextrins, glycolipids, glycosaminoglycans, amino acids (e.g.; but not limited to: glycine, sodium glutamate, proline, α-alanine, β-alanine, lysine-HCl, lysine, 4-hydroxyproline), peptides and polypeptides, proteins, amines (e.g.; but not limited to: betaine, trimethylamine N-oxide), lipo-proteic molecules, polyols, gums, waxes, antioxidants, anti-reductants, buffering agents, inorganic and organic salts (e.g; but not limited to: ammonium, sodium, and magnesium sulfate, potassium phosphate, sodium fluoride, sodium acetate, sodium polyethylene, sodium caprylate, propionate, lactate, succinate, PF6-succinate), radical scavengers, natural or synthetic polymers, a binder (e.g.; but not limited to: starch; gelatin; sugars as sucrose, glucose, dextrose, molasses, and lactose; natural and synthetic gums such as acacia, sodium alginate, extract of Irish moss, panwar gum, ghatti gum, mucilage of isapol husks, carboxymethylcellulose, methylcellulose, hydroxypropyl methylcellulose, hydroxylpropyl cellulose, ethyl cellulose, polyvinylpyrrolidone, Veegum, larch arabogalactan; polyethylene glycols; ethylcellulose; waxes; water and achools, amylase, methacrylate and methyl methacrylate copolymers), disaggregant (e.g.; but not limited to: starches, clays, celluloses, algins, gums, cross-linked natural and synthetic polymers, Veegum HV, methylcellulose, agar, bentonite, cellulose and wood products, natural sponge, cation-exchange resins, alginic acid, guar gum, citrus pulp, carboxymethylcellulose, combinations of sodium lauryl sulfate and starch) used in any of their physical or processed states. Any derivative of above-mentioned molecules are included as well.

In one embodiment, the compound is covalently associated with the gel, foam, film, or particles as described herein, or in another embodiment, the compound forms a physical interaction with the gel, foam, film, or particle.

In one embodiment, the compound is covalently associated with the gel, foam, film, or particle via the use of a cross-linking agent. In one embodiment, the term “cross-linking agent” refers to an agent which facilitates the formation of a covalent bond between 2 molecules. In one embodiment, the cross-linking agent is a zero-length cross-linking agent.

In one embodiment, the cross-linking agent is (1 ethyl 3-(3dimethyl aminopropyl)carbodiimide (EDAC), N-Sulfohydroxy succinamide (Sulfo NI-IS), EDC, WSC, 5-iodopyrimidines, N-carbalkoxydihydroquuinolines, pyrroloquinolinequinones, or a combination thereof.

In one embodiment, the cross-linking agent is a homobifunctional cross-linker, such as, for example, a N-hydroxysuccinimide ester (e.g. disuccinimidyl suberate or dithiobis(succinimidylpropionate), homobifunctional imidoester (e.g dimethyladipimidate or dimethyl pimelimidate), sulfhydryl-reactive crosslinker (e.g. 1,4-di-[3′-(2′-pyridyldithio)propionamido]butane), difluorobenzene derivative (e.g. 1,5-difluoro-2,4-dinitrobenzene), aldehyde (e.g. formaldehyde, glutaraldehyde), bis-epoxide (e.g. 1,4-butanediol diglycidyl ether), hydrazide (e.g., adipic acid dihydrazide), bis-diazonium derivative (e.g. o-tolidine), bis-alkylhalide, or a combination thereof.

In one embodiment, the cross-linking agent is a heterobifunctional cross-linker, such as, for example, an amine-reactive and sulfhydryl-reactive crosslinker (e.g. N-succinimidyl 3-(2-pyridyldithio)propionate, a carbonyl-reactive and sulfhydryl-reactive crosslinker (e.g. 4-(4-N-maleimidophenyl)butyric acid hydrazide), or a combination thereof.

In some embodiments, the cross-linking agent is a trifunctional cross-linkers, such as, for example, 4-azido-2-nitrophenylbiocytin-4-nitrophenyl ester, sulfosuccinimidyl-2-[6-biotinamido]-2-(p-azidobenzamido)hexanoamido]ethyl-1,3′-dithiopropionate (sulfo-SBED), or a combination thereof.

In another embodiment, the cross-linking agent is an enzyme, which in one embodiment, is a transglutaminase, peroxidase, xanthine oxidase, polymerase, ligase, or a combination thereof.

The choice of concentration of the cross-linking agent utilized for activity will vary, as a function of the volume, agent and polymer chosen, in a given application, as will be appreciated by one skilled in the art.

In one embodiment, the compound is associated with the gel, foam, film, or particle via physical association, such as, for example, via imbibing of any means, for example via the application of heat to promote the physical association. In another embodiment, the compound-associated gel, foam, film, or particle may be attached to a substrate via physical association, such as, for example, via imbibing of any means, for example via air drying of the gel, foam, film, or particle, or via the application of heat thereto, to promote the physical association, of via the use of a cross-linking agent as described herein. In one embodiment, the effector compound, or compound of interest is applied directly to the gels, foams, films, etc. of this invention. In one embodiment, the effector compound, or compound of interest is applied directly, without being dispersed in any solvent. In some embodiments, the effector compound solubility changes as a function of its association with a gel, foam, film or particle as herein described.

In some embodiments, the gels of this invention are applied in a dry form. In some embodiments, the gels of this invention are subjected to solvent removal, prior to use, for example, via lyophilization or by spray-drying. In one embodiment, application of heat will be at a temperature which promotes association of the compound thereto, however, does not diminish or significantly diminish activity of the compound.

In another embodiment, the effector compound is an antibiotic, an antiviral, an antifungal, an anti-helminth, an anti-inflammatory, an antihistamine, an immunomodulatory, an anticoagulant, a surfactant, a bronchodilator, an antibody, a beta-adrenergic receptor inhibitor, a calcium channel blocker, an ace inhibitor, a growth factor, a hormone, a DNA, an siRNA, a vector or any combination thereof.

In one embodiment, the term “effector compound” refers to any agent or compound, which has a specific purpose or application which is useful in the treatment, prevention, inhibition, suppression, delay or reduction of incidence of infection, a disease, a disorder, or a condition, when applied to the gels, foams, films, particles, compositions, kits and/or methods of this invention. An effector compound of this invention, in one embodiment, will produce a desired effect which is exclusive to the ability to image the compound. In some embodiments, the effector compound may be useful in imaging a site at which the compound is present, however, such ability is secondary to the purpose or choice of use of the compound.

In some embodiments, the of gels, foams, films, particles, compositions, kits and/or methods this invention make use of the incorporation of any compound of interest. Such gels, foams, films, particles, compositions, and/or kits may be utilized, in some embodiments, as a means delivery of the compound of interest to a desired site in a subject, or a means of imaging a site in a subject

In one embodiment, the term “compound of interest”, as used anywhere herein, refers to any desired molecule, and may comprise, inter alia, a nucleic acid, a hormone, a growth factor, a cytokine, a chemokine, a bone morphogenetic protein, a matrix metallo-proteinases, a peptide, a drug, an enzyme, a label or a combination thereof.

The term “effector compound” is to be understood to include the terms “drug” and “agent”, as well, when referred to herein, and represent a molecule whose incorporation within the gels, foams, films, particles, compositions, and/or kits of this invention, or whose use thereof, is desired. In one embodiment, the agent is incorporated directly within the gels, foams, films, particles, compositions, and/or kits of this invention or, in another embodiment, the agent is incorporated within the gels, foams, films, particles, compositions, and/or kits of this invention, either by physical interaction with the gels, foams, films, particles, compositions, and/or kits of this invention, or association thereto.

In one embodiment, compounds for use in gels, foams, films, particles, compositions, and/or kits of this invention and/or methods of this invention may comprise, inter-alia, an antibody or antibody fragment, a peptide, an oligonucleotide, a ligand for a biological target, an immunoconjugate, a chemomimetic functional group, a glycolipid, a labelling agent, an enzyme, a metal ion chelate, an enzyme cofactor, a cytotoxic compound, a bactericidal compound, a bacteriostatic compound, a fungicidal compound, a fungistatic compound, a chemotherapeutic, a growth factor, a hormone, a cytokine, a toxin, a prodrug, an antimetabolite, a microtubule inhibitor, a radioactive material, a targeting moiety, or any combination thereof. In some embodiments, the effector compound will comprise an ABC transporter inhibitor, an MDR inhibitor, other DNA moieties, siRNAs etc., quorum sensing molecules, lipids, or other molecules of interest.

In one embodiment, the term “antibody or antibody fragment” refers to intact antibody molecules as well as functional fragments thereof, such as Fab, F(ab′)2, and Fv that are capable of binding to an epitope. In one embodiment, an Fab fragment refers to the fragment which contains a monovalent antigen-binding fragment of an antibody molecule, which can be produced by digestion of whole antibody with the enzyme papain to yield an intact light chain and a portion of one heavy chain. In one embodiment, Fab′ fragment refers to a part of an antibody molecule that can be obtained by treating whole antibody with pepsin, followed by reduction, to yield an intact light chain and a portion of the heavy chain. Two Fab′ fragments may be obtained per antibody molecule. In one embodiment, (Fab′)2 refers to a fragment of an antibody that can be obtained by treating whole antibody with the enzyme pepsin without subsequent reduction. In another embodiment, F(ab′)2 is a dimer of two Fab′ fragments held together by two disulfide bonds. In one embodiment, Fv, may refer to a genetically engineered fragment containing the variable region of the light chain and the variable region of the heavy chain expressed as two chains. In one embodiment, the antibody fragment may be a single chain antibody (“SCA”), a genetically engineered molecule containing the variable region of the light chain and the variable region of the heavy chain, linked by a suitable polypeptide linker as a genetically fused single chain molecule.

Methods of making these fragments are known in the art. (See for example, Harlow and Lane, Antibodies: A Laboratory Manual, Cold Spring Harbor Laboratory, New York, 1988, incorporated herein by reference).

In one embodiment, compounds for use in the gels, foams, films, particles, compositions, and/or kits of this invention and/or methods of this invention may comprise, inter-alia, a peptide. In some embodiments, the term “peptide” refers to native peptides (either degradation products, synthetically synthesized peptides or recombinant peptides) and/or peptidomimetics (typically, synthetically synthesized peptides), such as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C A. Ramsden Gd., Chapter 17.2, F. Choplin Pergamon Press (1992), which is incorporated by reference as if fully set forth herein.

In one embodiment, the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” may include both D- and L-amino acids. In some embodiments, the term “amino acid” encompasses artificial amino acids or chemically protected amino acids.

In one embodiment, the gels, foams, films, particles, compositions, and/or kits of this invention and/or methods of this invention comprise or make use of an oligonucleotide, a nucleic acid, or a vector. In some embodiments, the term “oligonucleotide” is interchangeable with the term “nucleic acid”, and may refer to a molecule, which may include, but is not limited to, prokaryotic sequences, eukaryotic mRNA, cDNA from eukaryotic mRNA, genomic DNA sequences from eukaryotic (e.g., mammalian) DNA, and even synthetic DNA sequences. The term also refers to sequences that include any of the known base analogs of DNA and RNA.

The gels, foams, films, particles, compositions, and/or kits of this invention and/or methods of this invention may comprise nucleic acids, in one embodiment, or in another embodiment, the gels, foams, films, particles, compositions, and/or kits of this invention and/or methods of this invention may include delivery of the same, as a part of a particular vector. In one embodiment, polynucleotide segments encoding sequences of interest can be ligated into commercially available expression vector systems suitable for transducing/transforming mammalian cells and for directing the expression of recombinant products within the transduced cells. It will be appreciated that such commercially available vector systems can easily be modified via commonly used recombinant techniques in order to replace, duplicate or mutate existing promoter or enhancer sequences and/or introduce any additional polynucleotide sequences such as for example, sequences encoding additional selection markers or sequences encoding reporter polypeptides.

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product, such as an enzymatic assay. If the gene product of interest to be expressed by a cell is not readily assayable, an expression system can first be optimized using a reporter gene linked to the regulatory elements and vector to be used. The reporter gene encodes a gene product, which is easily detectable and, thus, can be used to evaluate efficacy of the system. Standard reporter genes used in the art include genes encoding β-galactosidase, chloramphenicol acetyl transferase, luciferase and human growth hormone.

As will be appreciated by one skilled in the art, a fragment or derivative of a nucleic acid sequence or gene that encodes for a protein or peptide can still function in the same manner as the entire, wild type gene or sequence. Likewise, forms of nucleic acid sequences can have variations as compared to wild type sequences, nevertheless encoding the protein or peptide of interest, or fragments thereof, retaining wild type function exhibiting the same biological effect, despite these variations. Each of these represents a separate embodiment of this present invention.

The nucleic acids can be produced by any synthetic or recombinant process such as is well known in the art. Nucleic acids can further be modified to alter biophysical or biological properties by means of techniques known in the art. For example, the nucleic acid can be modified to increase its stability against nucleases (e.g., “end-capping”), or to modify its solubility, or binding affinity to complementary sequences.

Methods for modifying nucleic acids to achieve specific purposes are disclosed in the art, for example, in Sambrook et al. (1989). Moreover, the nucleic acid sequences of the invention can include one or more portions of nucleotide sequence that are non-coding for the protein of interest. Variations in the DNA sequences, which are caused by point mutations or by induced modifications (including insertion, deletion, and substitution) to enhance the activity, half-life of production of the polypeptides encoded thereby, are also encompassed in the invention.

In one embodiment, the agent is one which may inhibit gene expression in a subject. In one embodiment, the agent that inhibits gene expression, activity or function comprises a nucleic acid. The nucleic acid may, in one embodiment, be DNA, or in another embodiment, the nucleic acid is RNA. In other embodiments, the nucleic acid may be single or double stranded.

In one embodiment, the agents used in the gels, foams, films, particles, compositions, and/or kits of this invention and/or methods of this invention may be used for gene silencing applications. In one embodiment, the activity or function of a particular gene is suppressed or diminished, via the use of antisense oligonucleotides. In one embodiment, the antisense molecules may be conjugated to the polymers of this invention. Inhibition of gene expression, activity or function is effected, in another embodiment, via the use of small interfering RNAs, which provides sequence-specific inhibition of gene expression for example, as described in Elbashir S M, et al (2001) Nature 411:494-498; Fire et al. (1998) Nature 391: 806-11; Waterhouse, P. M., et al. (1998). Proc. Natl. Acad. Sci. USA 95, 13959-13964 and Wang, Z., et al. (2000). J. Biol. Chem. 275, 40174-40179.

In some embodiments, transfected, transduced or transformed cells, may be incorporated into gels or compositions or materials of this invention, so that engineered cells may comprise the gels or compositions or materials of this invention.

In one embodiment, the nucleic acid encodes for an antibacterial, antiviral, antifungal or antiparasitic peptide or protein. In another embodiment, the nucleic acid encodes for a peptide or protein with cytotoxic or anti-cancer activity. In another embodiment, the nucleic acid encodes for an enzyme, a receptor, a channel protein, a hormone, a cytokine, a bone morphogenetic protein, a matrix metallo-proteinase, or a growth factor. In another embodiment, the nucleic acid encodes for a peptide or protein, which is immunostimulatory. In another embodiment, the nucleic acid encodes for a peptide or protein, which inhibits inflammatory or immune responses.

In one embodiment, gels, foams, films, particles, compositions, and/or kits of this invention and/or methods of this invention may further comprise or make use of a “drug” or “compound” or “agent”, which refers in some embodiments, to a substance applicable for use in the diagnosis, or in another embodiment, cure, or in another embodiment, mitigation, or in another embodiment, treatment, or in another embodiment, prevention, or in another embodiment, suppression, or in another embodiment, delay in progression, or in another embodiment delay or prevention of relapse, or in another embodiment, reduction in incidence of a disease, disorder, condition or infection. In one embodiment, the “drug” or “compound” or “agent” for use in the gels, foams, films, particles, compositions, and/or kits of this invention and/or methods of this invention this invention, refers to any substance which affects the structure or function of the target to which it is applied.

In another embodiment, the “drug” or “compound” or “agent” for use in the gels, foams, films, particles, compositions, and/or kits of this invention and/or methods of this invention, is a molecule that alleviates a symptom of a disease or disorder when administered to a subject afflicted thereof. In one embodiment, the “drug” or “compound” or “agent” for use in gels, foams, films, particles, compositions, and/of kits of this invention and/or methods of this invention, is a synthetic molecule, or in another embodiment, a naturally occurring compound isolated from a source found in nature.

In one embodiment, the “drug” or “compound” or “agent” for use in the gels, foams, films, particles, compositions, and/or kits of this invention and/or methods of this invention, may comprise antihypertensives, antidepressants, antianxiety agents, anticlotting agents, anticonvulsants, blood glucose-lowering agents, decongestants, antihistamines, histamine, antitussives, anti-inflammatories, antipsychotic agents, cognitive enhancers, cholesterol-reducing agents, antiobesity agents, autoimmune disorder agents, anti-impotence agents, antibacterial and antifungal agents, hypnotic agents, anti-Parkinsonism in agents, antibiotics, antiviral agents, anti-neoplastics, barbituates, sedatives, nutritional agents, beta blockers, emetics, anti-emetics, diuretics, anticoagulants, cardiotonics, androgens, corticoids, anabolic agents, growth hormone secretagogues, anti-infective agents, coronary vasodilators, carbonic anhydrase inhibitors, antiprotozoals, gastrointestinal agents, serotonin antagonists, anesthetics, hypoglycemic agents, dopaminergic agents, anti-Alzheimer's Disease agents, anti-ulcer agents, platelet inhibitors and glycogen phosphorylase inhibitors, insulin, diagnostic markers, drugs used fox the control of birth, natural products, calcyfying agents, cell mediators, cell inhibitors, antimitotic agents, alkylating agents, immunomodulators, analgesics, vaccines, sympathomimetic agents, cholinomimetic agents, adrenergic and adrenergic neuron blocking agent, antimuscarinic and antispasmodic agents, skeletal muscle relaxant, anti-migrane agents, central nervous system stimulans, immunosuppressive agents, vitamins, parasiticides, drugs for the treatment of ipo-/ipet-tytoidism, osteoporosis, osteopetrosis, arthritis, epilepsy, glaucoma and eye diseases.

In one embodiment, examples of the “thug” or “compound” or “agent” for use in the gels, foams, films, particles, compositions, and/or kits of this invention and/or methods of this invention comprise, inter-alia, antihypertensives including prazosin, nifedipine, trimazosin, amlodipine, and doxazosin mesylate; the antianxiety agent hydroxyzine; a blood glucose lowering agent such as glipizide; an anti-impotence agent such as sildenafil citrate; anti-neoplastics such as chlorambucil, lomustine or echinomycin; anti-inflammatory agents such as betamethasone, prednisolone, piroxicam, aspirin, flurbiprofen and (+)-N-{4-[3-(4-fluotophenoxy)phenoxy]-2-cyclopenten-1-yl}-N-hydroxyurea; antivirals such as acyclovir, nelfinavir, virazole; vitamins/nutritional agents such as retinol and vitamin E; emetics such as apomorphine; diuretics such as chlorthalidone and spironolactone; an anticoagulant such as dicumarol; cardiotonics such as digoxin and digitoxin; androgens such as 17-methyltestosterone and testosterone; a mineral corticoid such as desoxycorticosterone; a steroidal hypnotic/anesthetic such as alfaxalone; an anabolic agent such as fluoxymesterone or methanstenolone; antidepression agents such as fluoxetine, pyroxidine, venlafaxine, sertraline, paroxetine, sulpiride, [3,6-dimethyl-2-(2,4,6-trimethyl-phenoxy)-pyridin-4-yl]-(lethylpropyl)-amine or 3,5-dimethyl-4-(3′-pentoxy)-2-(2,4,6-trimethyl-phenoxy)pyridine; an antibiotic such as ampicillin and penicillin G or belonging to the family of penicillines, cephalosporins, aminoglycosidics, macrolides, carbapenem and penem, beta-lactam monocyclic, inhibitors of beta-lactamases, tetracyclins, polipeptidic antibiotics, chloramphenicol and derivatives, fusidic acid, lincomicyn, novobiocine, spectinomycin, poly-etheric ionophores, quinolones; an anti-infective such as benzalkonium chloride or chlorhexidine; a coronary vasodilator such as nitroglycerin or mioflazine; a hypnotic such as etomidate; a carbonic anhydrase inhibitor such as acetazolamide or chlorzolamide; an antifungal such as econazole, terconazole, fluconazole, voriconazole or griseofulvin; an antiprotozoal such as metronidazole; an imidazole-type anti-neoplastic such as tubulazole; an anthelmintic agent such as thiabendazole or oxfendazole; an antihistamine such as astemizole, levocabastine, cetirizine, or cinnarizine; a decongestant such as pseudoephedrine; antipsychotics such as fluspirilene, penfluridole, risperidone or ziprasidone; a gastrointestinal agent such as loperamide or cisapride; a serotonin antagonist such as ketanserin or mianserin; an anesthetic such as lidocaine; a hypoglycemic agent such as acetohexamide; an anti-emetic such as dimenhydrinate; an antibacterial such as cotrimoxazole; a dopaminergic agent such as L-DOPA; anti-Alzheimer agents such as THA or donepezil; an anti-ulcer agent/H2 antagonist such as famotidine; a sedative/hypnotic such as chlordiazepoxide or triazolam; a vasodilator such as alprostadil; a platelet inhibitor such as prostacyclin; an ACE inhibitor/antihypertensive such as enalaprilic acid or lisinopril; a tetracycline antibiotic such as oxytetracycline or minocycline; a macrolide antibiotic such as azithromycin, clarithromycin, erythromycin or spiramycin; and glycogen phosphorylase inhibitors such as [R—(R*S*)]-5-chloro-N-[2-hydroxy-3{methoxymethylamino}-3-oxo-1-(phenylmethyl)-propyl]-IH-indole-2-carboxamide or 5-chloro-1-Hindole-2-carboxylic acid [(IS)-benzyl(2R)-hydroxy-3-((3R,4S)dihydroxy-pyrrolidin-1-yl-)-oxypropyl]amide.

Further examples of the “drug” or “compound” of “agent” for use in the gels, foams, films, particles, compositions, and/or kits of this invention and/or methods of this invention, are the glucose-lowering drug chlorpropamide, the anti-fungal fluconazole, the anti-hypercholesterolemic atorvastatin calcium, the antipsychotic thiothixene hydrochloride, the anxiolytics hydroxyzine hydrochloride or doxepin hydrochloride, the anti-hypertensive amlodipine besylate, the antiinflammatories piroxicam and celicoxib and valdicoxib, and the antibiotics carbenicillin indanyl sodium, bacampicillin hydrochloride, troleandomycin, and doxycycline hyclate.

In another embodiment a “drug” or “compound” or “agent” for use in the gels, foams, films, particles, compositions, and/or kits of this invention and/or methods of this invention, may comprise other antineoplastic agents such as platinum compounds (e.g., spiroplatin, cisplatin, and carboplatin), methotrexate, fluorouracil, adriamycin, mitomycin, ansamitocin, bleomycin, cytosine arabinoside, arabinosyl adenine, mercaptopolylysine, vincristine, busulfan, chlorambucil, melphalan (e.g., PAM, L-PAM or phenylalanine mustard), mercaptopurine, mitotane, procarbazine hydrochloride dactinomycin (actinomycin D), daunorubicin hydrochloride, doxorubicin hydrochloride, paclitaxel and other taxenes, rapamycin, manumycin A, INP-470, plicamycin (mithramycin), aminoglutethimide, estramustine phosphate sodium, flutamide, leuprolide acetate, megestrol acetate, tamoxifen citrate, testolactone, trilostane, amsacrine (m-AMSA), asparaginase (L-asparaginase) Erwina asparaginase, interferon alpha-2a, interferon alpha-2b, teniposide (VM-26), vinblastine sulfate (VLB), vincristine sulfate, bleomycin sulfate, hydroxyurea, procarbazine, and dacarbazine; mitotic inhibitors such as etoposide, colchicine, and the vinca alkaloids, radiopharmaceuticals such as radioactive iodine and phosphorus products; hormones such as progestins, estrogens and antiestrogens; anti-helmintics, antimalarials, and antituberculosis drugs; biologicals such as immune serums, antitoxins and antivenoms; rabies prophylaxis products; bacterial vaccines; viral vaccines; respiratory products such as xanthine derivatives theophylline and aminophylline; thyroid agents such as iodine products and anti-thyroid agents; cardiovascular products including chelating agents and mercurial diuretics and cardiac glycosides; glucagon; blood products such as parenteral iron, hernia, hematoporphyrins and their derivatives; biological response modifiers such as muramyldipeptide, muramyltripeptide, microbial cell wall components, lymphokines (e.g., bacterial endotoxin such as lipopolysaccharide, macrophage activation factor), sub-units of bacteria (such as Mycobacteria, Corynebacteria), the synthetic dipeptide N-acetyl-muramyl-L-alanyl-D-isoglutamine; anti-fungal agents such as ketoconazole, nystatin, griseofulvin, flucytosine (5-fc), miconazole, Amphotericin B, ricin, cyclosporins, and β-lactam antibiotics (e.g., sulfazecin); hormones such as growth hormone, melanocyte stimulating hormone, estradiol, beclomethasone dipropionate, betamethasone, betamethasone acetate and betamethasone sodium phosphate, vetamethasone disodium phosphate, vetamethasone sodium phosphate, cortisone acetate, dexamethasone, dexamethasone acetate, dexamethasone sodium phosphate, flunisolide, hydrocortisone, hydrocortisone acetate, hydrocortisone cypionate, hydrocortisone sodium phosphate, hydrocortisone sodium succinate, methylprednisolone, methylprednisolone acetate, methylprednisolone sodium succinate, paramethasone acetate, prednisolone, prednisolone acetate, prednisolone sodium phosphate, prednisolone tebutate, prednisone, triamcinolone, triamcinolone acetonide, triamcinolone diacetate, triamcinolone hexacetonide, fludrocortisone acetate, oxytocin, vassopressin, and their derivatives; vitamins such as cyanocobalamin neinoic acid, retinoids and derivatives such as retinol palmitate, and alpha-tocopherol; peptides, such as manganese super oxide dismutase; enzymes such as alkaline phosphatase; anti-allergic agents such as amelexanox; anti-coagulation agents such as phenprocoumon and heparin; circulatory drugs such as propranolol; metabolic potentiators such as glutathione; antituberculars such as para-aminosalicylic acid, isoniazid, capreomycin sulfate cycloserine, ethambutol hydrochloride ethionamide, pyrazinamide, rifampin, and streptomycin sulfate; antivirals such as amantadine azidothymidine (AZT, DDI, Foscarnet, or Zidovudine), ribavirin and vidarabine monohydrate (adenine arabinoside, ara-A); antianginals such as diltiazem, nifedipine, verapamil, erythritol tetranitrate, isosorbide dinitrate, nitroglycerin (glyceryl trinitrate) and pentaerythritol tetranitrate; anticoagulants such as phenprocoumon, heparin; antibiotics such as dapsone, chloramphenicol, neomycin, cefaclor, cefadroxil, cephalexin, cephradine erythromycin, clindamycin, lincomycin, amoxicillin, ampicillin, bacampicillin, carbenicillin, dicloxacillin, cyclacillin, picloxacillin, hetacillin, methicillin, nafcillin, oxacillin, penicillin including penicillin G and penicillin V, ticarcillin rifampin and tetracycline; antiinflammatories such as diflunisal, ibuprofen, indomethacin, meclofenamate, mefenamic acid, naproxen, oxyphenbutazone, phenylbutazone, piroxicam, sulindac, tolmetin, aspirin and salicylates; antiprotozoans such as chloroquine, hydroxychloroquine, metronidazole, quinine and meglumine antimonate; antirheumatics such as penicillamine; narcotics such as paregoric; opiates such as codeine, heroin, methadone, morphine and opium; cardiac glycosides such as deslanoside, digitoxin, digoxin, digitalin and digitalis; neuromuscular blockers such as atracurium mesylate, gallamine triethiodide, hexafluorenium bromide, metocurine iodide, pancuronium bromide, succinylcholine chloride (suxamethonium chloride), tubocruarine chloride and vecuronium bromide; sedatives (hypnotics) such as amobarbital, amobarbital sodium, aprobarbital, butabarbital sodium, chloral hydrate, ethchlorvynol, ethinamate, flurazepam hydrochloride, glutethimide, methotrimeprazine hydrochloride, methyprylon, midazolam hydrochloride, paraldehyde, pentobarbital, pentobarbital sodium, phenobarbital sodium, secobarbital sodium, talbutal, temazepam and triazolam; local anesthetics such as bupivacaine hydrochloride, chloroprocaine hydrochloride, etidocaine hydrochloride, lidocaine hydrochloride, mepivacaine hydrochloride, procaine hydrochloride and tetracaine hydrochloride; general anesthetics such as droperidol, etomidate, fentanyl citrate with droperidol, ketamine hydrochloride, methohexital sodium and thiopental sodium; and radioactive particles or ions such as strontium, iodide rhenium and yttrium.

In one embodiment, the “drug” or “compound” or “agent” for use in the gels, foams, films, particles, compositions, and/or kits of this invention and/or methods of this invention is a therapeutic compound. In one embodiment, the therapeutic compound is a peptide, a protein or a nucleic acid. In another embodiment, the therapeutic compound is an antibacterial, antiviral, antifungal or antiparasitic compound. In another embodiment, the therapeutic compound has cytotoxic or anti-cancer activity. In another embodiment, the therapeutic compound is an enzyme, a receptor, a channel protein, a hormone, a cytokine or a growth factor. In another embodiment, the therapeutic compound is immunostimulatory. In another embodiment, the therapeutic compound inhibits inflammatory or immune responses.

In one embodiment, the term “therapeutic”, refers to a molecule, which when provided to a subject in need, provides a beneficial effect. In some cases, the molecule is therapeutic in that it functions to replace an absence or diminished presence of such a molecule in a subject. In one embodiment, the molecule is a nucleic acid coding for the expression of a protein is absent, such as in cases of an endogenous null mutant being compensated for by expression of the foreign protein. In other embodiments, the endogenous protein is mutated, and produces a non-functional protein, compensated for by the expression of a heterologous functional protein. In other embodiments, expression of a heterologous protein is additive to low endogenous levels, resulting in cumulative enhanced expression of a given protein. In other embodiments, the molecule stimulates a signalling cascade that provides for expression, or secretion, or others of a critical element for cellular or host functioning.

In another embodiment, the therapeutic molecule may be natural or non-natural insulins, amylases, proteases, lipases, kinases, phosphatases, glycosyl transferases, trypsinogen, chymotrypsinogen, carboxypeptidases, hormones, ribonucleases, deoxyribonucleases, triacylglycerol lipase, phospholipase A2, elastases, amylases, blood clotting factors, UDP glucuronyl transferases, ornithine transcarbamoylases, cytochrome p450 enzymes, adenosine deaminases, serum thymic factors, thymic humoral factors, thymopoietins, growth hormones, somatomedins, costimulatory factors, antibodies, colony stimulating factors, erythropoietin, epidermal growth factors, hepatic erythropoietic factors (hepatopoietin), liver-cell growth factors, interleukins, interferons, negative growth factors, fibroblast growth factors, transforming growth factors of the α family, transforming growth factors of the β family, gastrins, secretins, cholecystokinins, somatostatins, serotonins, substance P, transcription factors or combinations thereof.

In another embodiment, this invention also comprises incorporation of any toxic substance for therapeutic purpose. In one embodiment, the gels, foams, films, particles, compositions, and/or kits of this invention and/or methods of this invention, may incorporate an oligonucleotide encoding a suicide gene, which when in contact with diseased cells or tissue, is expressed within such cells. In one embodiment, the term “suicide gene” refers to a nucleic acid coding for a product, wherein the product causes cell death by itself or in the presence of other compounds. A representative example of a suicide gene is one, which codes for thymidine kinase of herpes simplex virus. Additional examples are thymidine kinase of varicella zoster virus and the bacterial gene cytosine deaminase, which can convert 5-fluorocytosine to the highly cytotoxic compound 5-fluorouracil.

Suicide genes may produce cytotoxicity by converting a prodrug to a product that is cytotoxic. In one embodiment, the term “prodrug” means any compound that can be converted to a toxic product for cells. Representative examples of such a prodrug is gancyclovir which is converted in vivo to a toxic compound by HSV-thymidine kinase. The gancyclovir derivative subsequently is toxic to cells. Other representative examples of prodrugs include acyclovir, FIAU [1-(2-deoxy-2-fluoro-β-D-arabinofuranosyl)-5-iodouracil], 6-methoxypurine arabinoside for VZV-TK, and 5-fluorocytosine for cytosine deaminase.

In another embodiment, the cytotoxic agent may comprise any agent that is detrimental to cells, such as, for example, taxol, cytochalasin B, gramicidin D, ethidium bromide, emetine, mitomycin, etoposide, tenoposide, vincristine, vinblastine, colchicin, doxorubicin, daunorubicin, dihydroxy anthracinedione, mitoxantrone, mithramycin, actinomycin D, 1-dehydrotestosterone, glucocorticoids, procaine, tetracaine, lidocaine, propranolol, and puromycin and analogs or homologs thereof.

In one embodiment the drug, agent or effector compound may comprise any compound of choice to suit a particular application. Such compounds may comprise, inter-alia, wound healing promotional agents, antiseptics, anti-infectives, tissue engineering compounds, recombinant products or constructs, hormones, growth factors, enzymes, cytokines, antibodies, anti-inflammatories, immune modulating compounds, immunosuppressant, antihypertensives, antidepressants, antianxiety agents, anticlotting agents, anticonvulsants, blood glucose-lowering agents, decongestants, antihistamines, antitussives, antipsychotic agents, cognitive enhancers, cholesterol-reducing agents, antiobesity agents, autoimmune disorder agents, anti-impotence agents, antibacterial and antifungal agents, hypnotic agents, anti-Parkinsonism agents, anti-neoplastics, barbituates, sedatives, nutritional agents, beta blockers, emetics, anti-emetics, diuretics, anticoagulants, cardiotonics, androgens, corticoids, anabolic agents, growth hormone secretagogues, coronary vasodilators, carbonic anhydrase inhibitors, antiprotozoals, gastrointestinal agents, serotonin antagonists, anesthetics, hypoglycemic agents, dopaminergic agents, anti-Alzheimer's disease agents, anti-ulcer agents, platelet inhibitors and glycogen phosphorylase inhibitors.

Further examples of the “drug” or “compound” or “agent” for use in the gels, foams, films, particles, compositions, and/or kits of this invention and/or methods of this invention, comprise a polyene antifungal, which in some embodiments is amphotericin B. In some embodiments, the polyene antifungal will comprise natamycin, timocidin, filipin, nystatin, amphotericin A, pimaricin, rimocidin, tetraenes; eurocidin, pentaenes; cryptocidin, mediocidin, hexaenes; candicidin, candidin, candimycin, hamycin, levorin or trichomycin. In some embodiments, twor or more antifungal compounds or other effector compounds as herein described are incoporated within the coatings to form the coated materials of this invention, and application into any embodiment of this invention.

Further examples of the “drug” or “compound” or “agent” for use in the gels, foams, films, particles, compositions, and/or kits of this invention and/or methods of this invention, comprise imidazole and triazole antifungal drugs, such as those that inhibit the enzyme cytochrome P450 14α-demethylase. In some embodiments, the imidazole and triazole antifungal drugs comprise Miconazole, Ketoconazole, Clotrimazole, Econazole, Bifonazole, Butoconazole, Fenticonazole, Isoconazole, Oxiconazole, Sertaconazole, Sulconazole, Tioconazole, Fluconazole, Itraconazole, Isavuconazole, Ravuconazole, Posaconazole, Voriconazole or Terconazole.

Further examples of the “drug” or “compound” or “agent” for use in the gels, foams, films, particles, compositions, and/or kits of this invention and/or methods of this invention, comprise squalene epoxidase inhibitors, which in some embodiments are allylamines. In some embodiments, the allylamines comprise Terbinafine, Amorolfin, Naftifine, Butenafine. Further examples of the “drug” or “compound” or “agent” for use in the gels, foams, films, particles, compositions, and/or kits of this invention and/or methods of this invention, comprise Echinocandins, Anidulafungin, Caspofungin Micafungin, ciclopirox olamine, Flucytosine, Griseofulvin, Haloprogin, Tolnaftate, Undecylenic acid, or any combination of antifungal compounds as herein described.

In some embodiments, a method of this invention is to be understood as comprising the treatment of any disease with a coated material comprising gels, foams, films, particles, compositions, and/or kits of this invention, for example, using coated particles, wherein these materials comprise at least one of the compounds described herein, for which such compounds are useful, e.g., treating cardiovascular disease with a formulation comprising a beta blocker, and/or antihypertensive, and/or vasodilator, and/or anticoagulant, etc., as will be appreciated by one skilled in the art. Such particles, in one embodiment, will be a part of a composition suited for a particular route of administration, such as an oral formulation, or in other embodiments, parenteral formulations, as will be appreciated by one skilled in the art.

In some embodiments, a method of this invention is to be understood as comprising the treatment of any disease with the gels, foams, films, particles, compositions, and/or kits of this invention. In some embodiments, such gels, foams, films, particles, and/or compositions may be applied via any means accepted in the art, for example, and in some embodiments, gels may be injected in a subject at a desired site, wherein subsequent solidification of such gel comprising the effector compound occurs in situ. Compositions may comprise any known in the art, and may be applied, for example, topically. It is to be understood that any of the gels, foams, films, particles, and/or compositions may be applied individually, in combination, or associated with a substrate, and applied via any applicable means, and is to be considered as part of this invention.

In another embodiment, the coated materials of this invention comprise an agent, which may be a radioactive agent, which in other embodiments, may include any radioisotope which is known in the art, used for example in diagnosing cancer, or in anti-tumor applications. Examples include, but are not limited to, indium-111, cobalt-60, technecium-99. Additionally, naturally occurring radioactive elements such as uranium, radium, and thorium which typically represent mixtures of radioisotopes, are suitable examples of a radioactive agent. In another embodiment, magnetic particles may be thus used, such as, for example, magnetic iron oxide particles. The metal ions are typically chelated with an organic chelating moiety.

In one embodiment, the gels, foams, films, particles, compositions, and/or kits of this invention comprise a polymer dispersed in a polar solvent. In some embodiments, the solvent will comprise an oxygenated organic solvents, which in some embodiments are of from 1-6, more usually from 1-4 carbon atoms, including alcohols, ethers and the like. In one embodiment, the polar solvent is water, dimethylsulfoxide, dimethylformamide, an alcohol, such as for example, 1-butyl alcohol, or propanol, or ethanol, methoxyethanol, benzyl alcohol, or methanol. In some embodiments, the polar solvent is tetrahydrofuran, ethyl acetate, methyl acetate, cyclohexanone, methyl ethyl ketone (MEK), nitrobenzene, benzonitrile, dioxane, nitroethane, pyridine, acetone, acetic acid, acetonitrile, formamide, an ionic liquid, or N-methylpyrrolidinone (NMP)

In one embodiment, the gels, foams, films, particles, compositions, and/or kits of this invention comprise a polymer dispersed in a slightly polar, or essentially non-polar solvent.

In one embodiment, the substrate is a part of, or in the form of a bead. In one embodiment, the substrate is a part of, or in the form of a microparticle. In one embodiment, the substrate is a part of or in the form of a nanoparticle. In one embodiment, the gels of this invention are in the form of a bead, and comprise an effector compound as herein described.

In one embodiment, the substrate is a part of, or in the form of a bandage. In one embodiment, the substrate is a part of, or in the form of a suture.

In one embodiment, the substrate is a part of, or in the form of a catheter. In one embodiment, the catheter is a PA, pericardial, pleural, urinary or intra-abdominal catheter. In another embodiment, the catheter is a coronary catheter, epidural catheters peripheral vascular catheter, or neuro-interventional microcatheter.

In one embodiment, the substrate is a part of, or in the form of a stent. In one embodiment, the stent may comprise, inter-alia, an endovascular, biliary, tracheal, gastrointestinal, urethral, ureteral, esophageal and/or coronary stent. In one embodiment, the stent may comprise, inter-alia, a stent in the airway, hepatobiliary tract, and others, as will be appreciated by one skilled in the art.

In another embodiment, the substrate is a part of or in the form of an embolic coil, endovascular graft, guide wire, stylets, introducers, and/or balloon, and the like. In one embodiment, the balloon may comprise, inter-alia, a coronary balloon, peripheral vascular balloon, and/or neurological balloon.

In some embodiments, the coated stents include, for example, vascular stents such as self-expanding stents and balloon expandable stents. Examples of self-expanding stents useful in the present invention, and representing embodiments thereof, are in U.S. Pat. Nos. 4,655,771; 4,954,126; 5,061,275. In some embodiments, the stent which may be coated and comprise embodiments of the invention, or are for use according to the methods of this invention include, for example, an express stent such as the Express™ stent or an Express2™ stent.

The coated substrates, materials and/or devices of this invention may comprise metallic, ceramic, or polymeric materials, or a combination thereof.

In some embodiments, the metallic materials include metals and alloys based on titanium (such as nitinol, nickel titanium alloys, thermo-memory alloy materials), stainless steel, tantalum, nickel-chrome, or certain cobalt alloys including cobalt-chromium-nickel alloys such as Elgiloy® and Phynox®. Metallic materials also include clad composite filaments, such as those disclosed in WO 94/16646.

In some embodiments, the ceramic materials include, but are not limited to, oxides, carbides, or nitrides of the transition elements such as titaniumoxides, hafnium oxides, iridiumoxides, chromium oxides, aluminum oxides, and zirconiumoxides. Silicon based materials, such as silica, may also be used. Any of these materials may comprise a substrate or a part of a device of this invention, and may be coated with the gels, foams, films, compositions comprising effector compounds, as herein described.

In some embodiments, the coated substrates, materials and/or devices of this invention may comprise other compounds, including inter-alia, sterols such as cholesterol, stigmasterol, β-sitosterol, and estradiol; cholesteryl esters such as cholesteryl stearate; C12-C24 fatty acids such as lauric acid, myristic acid, palmitic acid, stearic acid, arachidic acid, behenic acid, and lignoceric acid; C18-C36 mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C16-C18 fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; sphingomyelins such as stearyl, palmitoyl, and tricosanyl sphingomyelins; ceramides such as stearyl and palmitoyl ceramides; glycosphingolipids; lanolin and lanolin alcohols; and combinations and mixtures thereof. Preferred non-polymeric materials include cholesterol, glyceryl monostearate, glycerol tristearate, stearic acid, stearic anhydride, glyceryl monooleate, glyceryl monolinoleate, and acetylated monoglycerides. The use of such materials will suit a particular application toward which the coated substrate, material and/or device is applied, as will be appreciated by one skilled in the art.

In some embodiments, the coating of a substrate, material and/or device will provide characteristics to the substrate, material and/or device to the particular application of use. For example, the coating of surgical instruments may comprise, inter-alia, incorporation of a compound which prevents or mitigates adesion to the surface of such instruments of biological material. In another embodiment, the coating of suturing material, or compositions which comprise a surgical glue, using for example the gels, foams, films and/or particles of this invention, may incorporate, inter-alia, a compound which promotes adhesion of, for example, skin flaps at a site of surgical incision.

In one embodiment, the substrate is a part of, or in the form of a valve. In one embodiment, the substrate is a part of, or in the form of a pacemaker. In one embodiment, the substrate is a part of, or in the form of a conduit. In one embodiment, the substrate is a part of, or in the form of a cannula.

In one embodiment, the substrate is a part of, or in the form of an appliance.

In one embodiment, the substrate is a part of, or in the form of a tissue scaffold.

In one embodiment, the substrate is a part of, or in the form of a central line.

In one embodiment, the substrate is a part of, or in the form of a pessary.

In one embodiment, the substrate is a part of, or in the form of a tube. In one embodiment, the tube is a tracheostomy, gastostomy tube, Γ-tube, enteral feeding device, endotracheal or chest tube.

In one embodiment, the substrate is a part of, or in the form of a drain. In one embodiment, the substrate is a part of, or in the form of a trochar or plug. In one embodiment, the drain is a cerebrospinal fluid drain.

In another embodiment, the substrate is a part of, or in the form of an implant. In one embodiment, the substrate is a part of, or in the form of a rod. In one embodiment, the substrate is a part of, or in the form of a screw. In one embodiment, the substrate is a part of, or in the form of an orthopedic appliance.

In another embodiment, the invention provides for coated implants, which may include, but are not limited to, vascular grafts, soft and hard tissue prostheses including, but not limited to, pumps, electrical devices including stimulators and recorders, auditory prostheses, artificial larynx, dental implants, mammary implants, penile implants, cranio/facial tendons, artificial joints, tendons, ligaments, menisci, and disks, artificial bones, artificial organs including artificial pancreas, artificial hearts, artificial limbs, and heart valves.

In another embodiment, the substrate is a part of, or in the form of a contraceptive device. In one embodiment, the substrate may be a part of, or in the form of, a diaphragm, a condom, a cervical cap, and the like. According to this aspect of the invention, the effector compound may comprise a spermicide, an antifungal, an antibiotic, an antiviral, a contraceptive vaccine, a contraceptive compound or a combination thereof

In another embodiment the substrate is a part of, or in the form of a product used for feminine hygeine. In one embodiment, such a product may include, inter-alia, a tampon, a padding, including sanitary napkin padding or nursing padding. In some embodiments, effector compounds incorporated into the coated feminine hygeine product may include, inter-alia, an antifungal, an antibiotic, an antiviral, an antiinflammatory, an analgesic, or a combination thereof.

In another embodiment, the coated materials of this invention may comprise, be in the form of, or be a part of tracheal devices, such as endotracheal tubes, aspirating devices and other tracheal suction devices, bronchoalveolar lavage catheters.

This invention provides, in some embodiments, coated materials which come into contact with human tissue. According to this aspect, and in one embodiment, the coating will be with compounds to suit a particular purpose, thus, for example, coating of a surgical material, such as, surgical instruments, suture material, implantable material, etc., may be coated with agents which are associated with successful completion of the surgery, such as, for example, anti-infective agents to minimize infection induced as part of the surgical procedure, or in another embodiment, agents which promote wound healing at the site of surgical intervention, or in another embodiment, an agent which addresses the therapeutic purpose of the surgery, for example, an anticancer compound administered at a site of surgical removal of a tumor, and others, as will be readily appreciated by one skilled in the art.

In some embodiments, such materials for which coating is envisaged will include, inter-alia, surgical, medical or dental instruments, bandages, patches, prosthesis, appliances, implants, scaffolding, suturing material, valves, pacemaker, stents, catheters, rods, shunt, tubing, wiring, electrodes, clips or fasteners, monitors, e.g., fetal monitors, contraceptive devices, feminine hygiene products, casting, endoscopes, and any others, which come into contact with human tissue In one embodiment, the materials comprise any surface that can be recharged with the compound of interest, such as, for example, recharging with antifungal molecules.

In some embodiments, the coated materials which come into contact with human tissue or fluids, comprise, for example, tissue transplants, pharmaceutical or cosmetic formulations, surgical glues or cements.

In one embodiment, the coated material may be affixed, glued, or sutured to the skin, or pierce the skin, or in another embodiment, the coated material serves as a portal through which other coated materials, or in another embodiment, non-coated materials, are passed through the skin.

In another embodiment, the substrate is a part of, or in the form of a pipe lining, a reactor, or equipment which comes into contact with food or seawater or wastewater. In some embodiments, the substrate is a part of, or in the form of a transparent viewing material, such as a windshield of a vehicle which moves on land, in air, or in seawater, wherein such coating provides characteristics to the material which are desirable, for example, improving visibility by diminishing glare, or fog, or diminishing water beading or adhesion, or covering of such device, or diminishing friction during movement, thereby enhancing speed, etc.

It is to be understood that any material, used for any purpose, to which the gels, foams, films, particles, and/or compositions of this invention may be applied, or which make use of the kits and/or methods of this invention, are to be considered embodiments of this invention.

In one embodiment, the coated materials of this invention incorporate an effector compound, which will promote, initiate or augment cell adhesion, thrombogenicity, healing, resolution of infection, resolution of a neoplastic or preneoplastic event, dilation or constriction of a vessel, etc., as will suit the application for which the material is used. In another embodiment, the coated materials of this invention incorporate an effector compound, which will mitigate, prevent or abrogate inflammation, hemolysis, bacterial and fungal adhesion and/or infection, unwanted mineral deposit, pain, etc., as will suit the application for which the material is used.

In some embodiments, the effector compound is a preservative, biocide, pesticide, anti-fouling agent, germicide, disinfectant, bio-effecting agent, algicide, vitamin, therapeutic agent or a combination thereof, applied at a therapeutic quantity and release time/concentration profile. Release time and concentration can be optimized by choice of coating agent, material, size, etc., as will be appreciated by one skilled in the art.

In one embodiment, the compound is realeased slowly, over a course of time, or in another embodiment, the compound is minimally released over a course of time.

In another embodiment, this invention provides a process for preparing a coated material comprising an effector compound covalently attached thereto, said process comprising the steps of:
a. preparing a gel or film comprising a polymer;
b. chemically reacting said gel or film with said compound; and
c. attaching said gel or film in (b) to at least a portion of a surface of a substrate.

In another embodiment, this invention provides a process for preparing a coated material comprising an effector compound covalently attached thereto, said process comprising the steps of:
a. preparing a foam or particle comprising a polymer;
b. chemically reacting said foam or particle with said compound; and
c. attaching said foam or particle in (b) to at least a portion of a surface of a substrate.

In one embodiment, reacting comprises activating the gel, film, foam or particle, etc., to produce an active ester, amine- or thiol-reactive group or photoreactive group in the gel, film, foam or particle, etc. In one embodiment, the ester is N-hydroxy-succinimide ester

In one embodiment, the term “activating” or “activated” when in reference to the preparation of any of the gels, films, foams or particles, etc., of this invention includes the formation of any type of chemical interaction between the gel, foam, film, particle, etc. and a desired material, for example, the substrate or compound. In some embodiments, the term “activating” prepares the stated material, e.g the gel, foam, film, particle for formation of a hydrogen bond, covalent bond, van der Walls interaction, p-p interaction, etc., with a substrate or compound, or in another embodiment, vice versa. In some embodiments, the term “activating” encompasses the creation of specific reactive species within the material whose formation of an interaction with another material is desired.

One embodiment of the preparation of a gel of this invention is exemplified hereinbelow, where a dextran prepolymer was reacted with Amyl-PEG-NHS to create a hydrogel, whose surface was then further modified to incorporate amphtericin B (AmB). Another embodiment of the gel preparation, does not require physical bond formation with the effector compound, in this case amphotericin B (AmB). In this embodiment, and as exemplified hereinbelow, dipping the hydrogel into a solution comprising DMF and amphotericin B overnight resulted in incorporation of AmB, which did not leak out appreciably over time.

In one embodiment, the gels of this invention are prepared such that polymerization and/or gel formation occurs in situ, following administration to a subject.

In one embodiment, attaching the gel, film, foam, particles, etc to the coated material is via chemically reacting said gel, film, foam, particles, etc. with at least a portion of a surface of said coated material. In another embodiment, attaching comprises activating at least a portion of a surface of the substrate, and providing conditions whereby the gel, film, foam or particle reacts with the activated surface of the substrate.

In another embodiment, this invention provides a process for preparing a coated material comprising an effector compound associated thereto, the process comprising the steps of:
a. preparing a gel or film comprising a polymer;
b. loading the gel or film with the compound; and
c. attaching the gel or film in (b) to at least a portion of a surface of a substrate.

In another embodiment, this invention provides a process for preparing a coated material comprising an effector compound associated thereto, the process comprising the steps of:
a. preparing a foam comprising a polymer;
b. loading the foam with the compound; and
c. attaching the foam in (b) to at least a portion of a surface of said coated material.

In another embodiment, the coated material is a coated particle, as described herein. In one embodiment, the coated particle comprises at least one polymer or block copolymer to which another polymer is attached, and the effector compound is bonded to or adsorbed onto the polymer-coated particle. In one embodiment, the particles prepared according to the methods of this invention will comprise a core, which is of a different material than the polymer which is attached to the core. Such particles are readily produced by methods well known in the art. In one embodiment, such particles may comprise a magnetic core, to which a polymer is attached, wrapped around, or otherwise associated, as will be understood by one skilled in the alt. An effector compound, or compound of interest may then be associated thereto, as herein described. According to this aspect, and in one embodiment, a magnet may be employed for the separation and/or retrieval of the prepared particles of this invention, for washing unbound effector compound from the particle, removal of unattached particles from the surface of the coated material, or a combination thereof.

In some embodiments the attachment of the gel, film, foam or particle to the surface of the material is accomplished via first imbibing a multi-functional monomer onto the portion of the surface of the material or device, or in another embodiment, the entire surface of the device or material. In one embodiment, the term “imbibing” refers to the multi-functional monomer being chemically or mechanically bonded to a polymeric surface.

In some embodiment, “monomer” refers to any material capable of polymerizing to or cross-linking with a polymer and can include monomers, oligomers, polymers, and the like.

In some embodiments, once the multi-functional monomer is bonded to the surface of the material, e.g., the medical device, the surface is contacted with a prepolymer, such as a hydrogel prepolymer.

According to this aspect of the invention, and in one embodiment, polymerization is then initiated causing the prepolymer to form into a hydrogel-polymer coating. During polymerization, the hydrogel polymer reacts with the multi-functional monomer. For instance, in one embodiment, the multi-functional monomer cross-links with the hydrogel polymer. In this manner; the multi functional monomer becomes part of the hydrogel polymer structure while simultaneously attaching the hydrogel polymer to the surface of the material, e.g. a device. According to this aspect and in one embodiment, a coating is formed on the surface of the material, e.g. device that is securely affixed thereto.

According to this aspect of the invention, and in one embodiment, the effector compound is associated with the gel, foam, film, or particle, etc. prior to its attachment to the surface of the material/device. In another embodiment, the compound is associated with the gel following its attachment to the surface of the material/device.

In one embodiment, the term “device” refers to a complete device or any part or component thereof. For example, in many applications, a part for a device will be treated in accordance with the present invention and then later assembled into the device.

In some embodiments, the coated devices of this invention can be made from any suitable thermoplastic or thermosensitive polymer capable of forming a mechanical or chemical attachment to the polymer, or in another embodiment, multi-functional monomer, and assembled as described, and/or as is known in the art. Suitable polymers include, for instance, silicones and urethanes. In one embodiment, the substrate, material or device can be made from polyvinyl chloride.

In order to attach the polymer or multi-functional monomer to the surface of the polymeric substrate, material or device, in some embodiments, the substrate, material or device is also contacted with a solvent that; is capable of solvating or swelling the polymer. The solvent and the multi functional monomer can be first combined together and then contacted with the medical device or can contact the medical device sequentially. In one embodiment, the solvent partially dissolves the surface of the substrate, material or device or otherwise causes the polymeric surface to swell. During swelling and partially dissolving, the multi-functional monomer can form a mechanical interlock with the surface. In other embodiments, the multi-functional monomer can also undergo a chemical reaction with the surface of the polymer.

Various solvents can be used in accordance with the present invention, as will be appreciated by one skilled in the art.

In some embodiments, the solvent is polar, or slightly polar. In some embodiments, the solvent is non-polar, or essentially non-polar.

Such solvents may include, inter-alia, dimethylsulfoxide (DMSO), acetone, alcohols, methylethyl ketone, toluene, xylene, N,N-dimethyl formamide (DME), tetrahydrofuran and the like. In some embodiments, the solvent is water.

The particular solvent chosen for an application will depend upon the type of polymer being coated and the type multi-functional monomer used.

For example, in some embodiments, when the substrate, material or device contains polyvinyl chloride, the solvent may be DMSO and ketones. If the substrate, material or device contains a urethane, in some embodiments, the solvent may be DMF or tetrahydrofuran. If the substrate, material or device contains silicone, in some embodiments, the solvent chosen may be toluene or xylene.

The multi-functional monomer used in embodiments of the present invention should be capable of mechanically or chemically bonding to the surface of the substrate, material or device and reacting with the gel, foam, film, or particle comprising a polymer, for example, a hydrogel polymer that is formed on the surface of the substrate, material or device. For example, and in one embodiment, a multi-functional monomer can be used that will cause cross-linking in a quaternary amine acrylate hydrogel polymer.

The multi-functional monomer can be, for instance, an acrylate such as a cationic quaternary ammonium monomer, other ammonium compounds, an acrylamide, or a vinyl pyrrolidone The multi-functional monomer may be bifunctional, trifunctional, tetrafunctional, pentafunctional, or hexafunctional.

Difunctional monomers which may be used, in some embodiments of the present invention include butylene glycol diacrylate, butylene glycol dimethacrylate, butanediol diacrylate, butanediol dimethacrylate, hexanediol diacrylate, hexanediol dimethacrylate, aliphatic dimethacrylate monomer, alkoxylated aliphatic diacrylate, alkoxylated cyclohexane dimethanol diacrylate, alkoxylated hexanediol diacrylate, alkoxylated; neopentyl glycol diacrylate, aromatic dimethacrylate monomer, caprolactione modified neopentylglycol hydroxypivalate diacrylate, cyclohexane dimethanol diacrylate, cyclohexane dimethanol dimethacrylate, diethylene glycol diacrylate, diethylene glycol dimethacrylate, dipropylene glycol diacrylate, ethoxylated (10) bisphenol a diacrylate, ethoxylated (2) bisphenol a dimethacrylate, ethoxylated (3) bisphenol a diacrylate, ethoxylated (30) bisphenol a diacrylate, ethoxylated (30) bisphenol a dimethacrylate, ethoxylated (4) bisphenol a diacrylate, ethoxylated (4) bisphenol a dimethacrylate, ethoxylated (8) bisphenol a dimethacrylate, ethoxylated bisphenol a dimethacrylate, ethoxylated bisphenol a dimethacrylate, ethoxylated (10) bisphenol dimethacrylate, ethoxylated (6) bisphenol a dimethacrylate, ethylene glycol dimethacrylate, hydroxypivalaldehyde modified trimethylolpropane diacrylate, neopentyl glycol diacrylate, neopentyl glycol I dimethacrylate, poly(ethylene glycol) (200) diacrylate, poly(ethylene glycol) (400) diacrylate, poly(ethylene glycol) (400) dimethacrylate, poly(ethylene glycol) (600) diacrylate, poly(ethylene glycol) (600) dimethacrylate, poly(ethylene glycol) dimethacrylate, poly(propylene glycol) dimethacrylate, propoxylated (2) neopentyl glycol diacrylate, tetraethylene glycol diacrylate, tetraethylene glycol dimethacrylate, triethylene glycol diacrylate, triethylene glycol dimethacrylate, tripropylene glycol diacrylate, and the like. The difunctional monomers are commercially available, for example, they may be purchased from the Sartomer Company, Inc. of Exton, Pa.

Trifunctional monomers that may be used in some embodiments of the present invention include ethoxylated (15) trimethylolpropane triacrylate, ethoxylated (3) trimethylolpropane triacrylate, ethoxylated (6) trimethylolpropane triacrylate, ethoxylated (9) trimethylolpropane triacrylate, ethoxylated (20) trimethylolpropane triacrylate, highly propoxylated (5.5) glyceryl triacrylate, low viscosity trimethylolpropane triacrylate, pentaerythritol triacrylate, propoxylated (3) glyceryl triacrylate, propoxylated (3) trimethylolpropane triacrylate, propoxylated (6) trimethylolpropane triacrylate, trimethylolpropane triacrylate, trimethylolpropane trimethacrylate, tris (2-hydroxy ethyl)isocyanurate triacrylate, and the like. Trifunctional monomers are commercially available, as well, and readily obtained, as will be understood by one skilled in the art.

Tetrafunctional, pentafunctional and hexafunctional monomers that may be used in some embodiments of the present invention include di-trimethylolpropane tetraacrylate, dipenteerythritol penteacrylate, ethoxylated (4) penteerythritol tetreacrylate, low; viscosity dipentaerythritol pentaacrylate, pentaacrylate ester, pentaerythritol tetraacrylate, caprolactone modified dipentaerythritol hexaacrylate, and the like.

In some embodiments, the monomers are acrylates, which may be alkoxylated using, for example, ethoxylate groups or propoxylate groups. Other acrylates which may be utilized include cationic quaternary ammonium monomers.

Examples of cationic quaternary ammonium monomers include N,N-dimethylaminoethyl acrylate DMS (dimethyl sulfate), N,N-dimethylaminoethyl acrylate MC (methyl chloride), N,N-dimethylaminoethyl methacrylate DMS, N,N-dimethylaminoethyl methacrylate MC, or diallyidimethylammonium chloride, which are commercially available, for example, from Ciba Specialty Chemicals. Other quaternary I ammonium monomers that may be used include acryloxethyidimethyl benzyl ammonium chloride, acryloxyethyltrimethyl ammonium chloride, methacryloxyethyidimethyl benzyl ammonium chloride, or methacryloxyethyltrimethyl ammonium chloride, which may be obtained commercially from Atofina.

Other examples of multi-functional monomers that can be used, in some embodiments of the present invention include methylene-bis-acrylamide (MBA) and diethylene glycol diacrylate, which are both commercially available from Polysciences, Inc, Warrington, Pa. Additional examples of multi-functional monomers which may be acceptable for use in the present invention include ethylene glycol diacrylate, triethylene glycol-bis-methacrylate, ethylene glycol-bis-methacrylate, ethylene glycol-di methacrylate, bisacrylamide, triethyleneglycol-bis-acrylate, 3,3′-ethylidene-bis(N-vinyl-2-pyrrolidone), trimethylolpropane trimethacrylate, glycerol trimethacrylate, poly(ethylene glycol) dimethacrylate, and other polyacrylate and polymethacrylate esters.

In addition to the solvent and the multi-functional monomer, the surface of the substrate, material or device can also be contacted with an initiator. For example, the initiator can be used to initiate polymerization of the hydrogel polymer that is to be formed on the surface.

Examples of initiators which may be used, in some embodiments, include, for example, IRGACURE(g) 184 (1-hydroxycyclohexyl phenyl ketone), and DAROCURE) 1173 (a-hydroxy-1, adimethylacetophenone) which are both commercially available from Ciba-Geigy Corp. These UV catalysts may be desirable, in some embodiments, because they are non-yellowing. Additional examples of initiators (which may be photo initiators or thermal initiators) may include, benzoyl peroxide, azo-bis-isobutyro nitrile, di-t-butyl peroxide, bromyl peroxide, cumyl peroxide, lauroyl peroxide, isopropyl percarbonate, methylethyl ketone peroxide, cyclohexane peroxide, t-butylhydroperoxide, di-t-amyl peroxide, dicymyl peroxide, t-butyl perbenzoate, Benzoin alkyl ethers (such as benzoin, benzoin isopropyl ether, and benzoin isobutyl ether), benzophenones (such as benzophenone and methyl-o-benzoyl benzoate), acetophenones (such as acetophenone, trichloroacetophenone, 2,2 diethoxyacetophenone, p-t-butyltrichloro-acetophenone, 2,2-dimethoxy-2-I phenylacetophenone, and p-dimethylaminoacetophenone), thioxanthones (such as xanthone, thioxanthone, 2-chlorothioxanthone, and 2-isopropyl thioxanthone), benzyl 2-ethyl anthraquinone, methylbenzoyl formate, 2-hydroxy-2-methyl-1 phenyl propane-1-one, 2-hydroxy-4′-isopropyl-2-methyl propiophenone, e-hydroxy ketone, tet-remethyl thiuram monosulfide, ailyl diazonium salt, and a combination of camphor quinone or 4-(N,N-dimethylamino)benzoate.

The substrate, material or device may be contacted, in some embodiments, with a solution comprised of one or more of the following components: a solvent, a multi-functional monomer, and an initiator, or the substrate, material or device may be contacted with the separate components in sequential steps. In one particular embodiment, a solution can be formed containing the solvent, the multi-functional monomer, and the initiator. The multi-functional monomer can be present in the solution in an amount from about 5% to about 50% by weight. The initiator can be present in the solution in an amount from about 0.05% to about 5.0% by weight. The substrate, material or device can be contacted with the solution, such as being dipped in the solution. In particular, the surface of the substrate, material or device can be contacted with the solution in an amount of time sufficient for the polymeric surface to either swell and/or partially dissolve. For example, the surface of the substrate, material or device optionally may be contacted with solution at room temperature for about 30 seconds to about 3 minutes.

If contacted with the solution, the substrate, material or device may be dried if desired although this step is not necessary For instance, the substrate, material or device can be heated or can simply be air dried. In this manner, the multi-functional monomer becomes imbibed into the surface of the polymer.

Also optional, polymerization may be initiated in a portion of the multi-function monomer. Partially polymerizing the multi-functional monomer may serve to create a better interlock with the surface of the substrate, material or device. Further, only a portion of the multi-functional monomer may be polymerized in order to leave active functional sites remaining. In some embodiments, it should be understood that partial polymerization of the multi-functional monomer may not be necessary.

Once the multi-functional monomer is imbibed into the surface of the substrate, material or device and mechanically or chemically attached to the substrate, material or device, the surface of the substrate, material or device is then contacted with a monomer or pre-polymer capable of forming, for example, the hydrogel polymer.

In another embodiment, this invention provides a method of preventing, diminishing or reducing the incidence of infection caused by introduction or implantation of a coated material in a subject, the method comprising attaching to a portion of a surface of said coated material, a gel or film comprising a polymer dispersed in a polar solvent comprising at least one effector compound, wherein said effector compound is associated with the prevention, diminishment or reduction in incidence of infection.

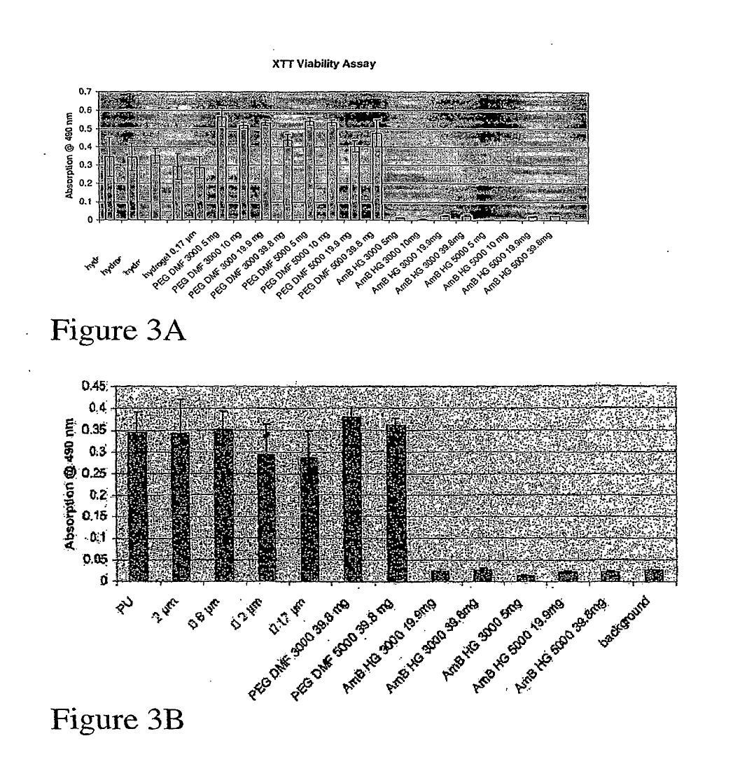

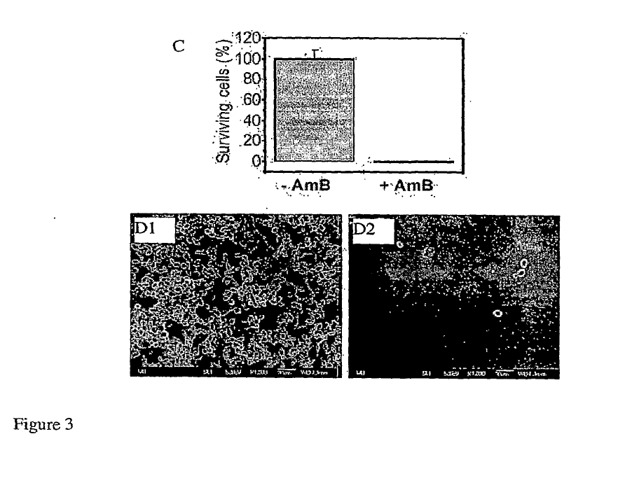

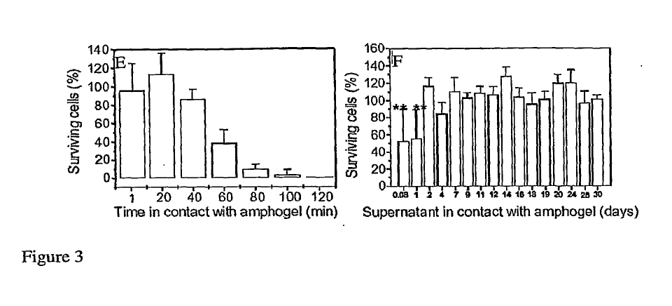

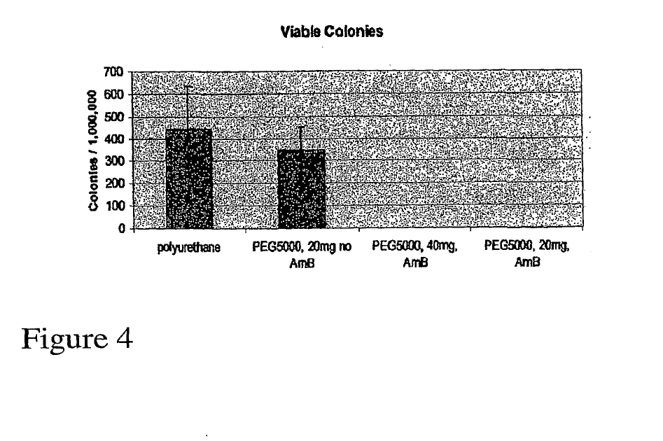

Anti-infective activity of the coated materials of this invention was exemplified herein in Example 1. Amphotericin B-modified polymer disks, incubated with Candida albicans led to almost complete killing of the Candida in XTT assays (FIG. 3) and a complete absence of colony counts obtained for these cultures (FIG. 4).

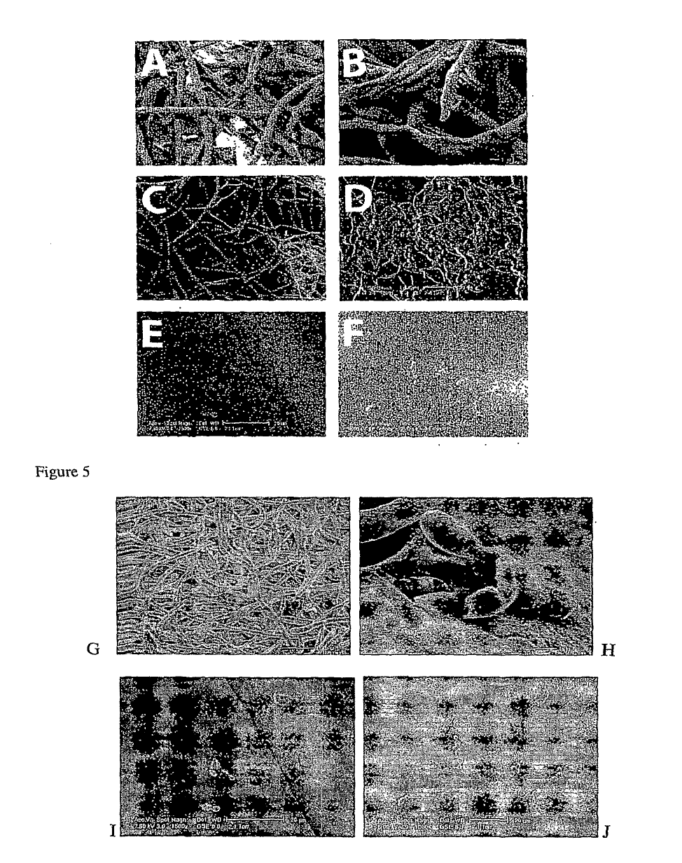

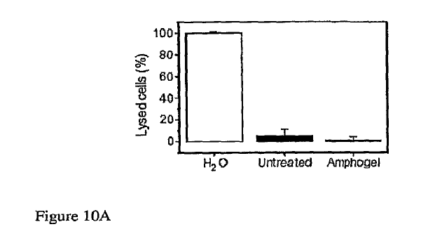

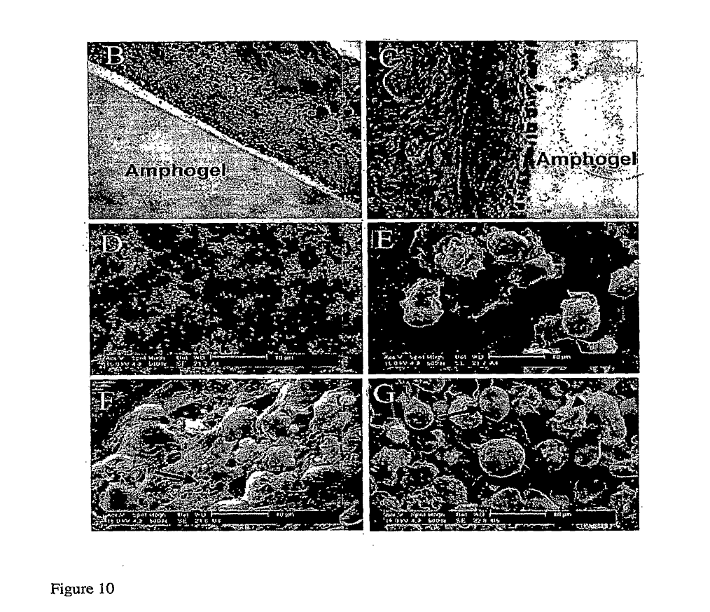

In some embodiments of this invention, the methods of this invention include the eradication, mitigation or control of biofilm formation on a surface. Such activity was exemplified with a coated material of this invention in Example 1 hereinbelow. Scanning electron microscopy of Amphotericin B-modified polymer disks and vehicle controls demonstrated formation of Candida biofilms on untreated polymeric surfaces, yet an absence of hyphae-formation on the treated disks in vitro (FIG. 5) and in vivo (FIG. 10).

In another embodiment, this invention provides a method of preventing, diminishing or reducing the incidence of infection caused by introduction or implantation of a coated material in a subject, the method comprising attaching to a portion of a surface of said coated material, a foam or particle comprising a polymer dispersed in a polar solvent comprising at least one effector compound, wherein said effector compound is associated with the prevention, diminishment or reduction in incidence of infection.

In another embodiment, the invention provides a method of preventing, diminishing or reducing the incidence of local fungal infection in a subject, said method comprising contacting a site of, or predisposed to infection with:
a. a substrate
b. a gel or film comprising a polymer attached thereto, on at least a portion of a surface of said substrate; and
c. at least one effector compound associated with said gel or film;
   wherein said at least one effector compound is associated with the prevention, diminiution or reduction of the incidence of said fungal infection.

In another embodiment, the invention provides a method of preventing, diminishing or reducing the incidence of local fungal infection in a subject, said method comprising contacting a site of, or predisposed to infection with:
a. a substrate
b. a foam or particle comprising a polymer attached thereto, on at least a portion of a surface of said substrate; and
c. at least one effector compound associated with said foam or particle;
   wherein said at least one effector compound is associated with the prevention, diminution or reduction of the incidence of said fungal infection.

In another embodiment, this invention provides a method of preventing, diminishing or reducing the incidence of infection in a subject, the method comprising administering to the subject a gel, foam, film or composition of this invention comprising at least one effector compound, wherein said effector compound is associated with the prevention, diminishment or reduction in incidence of infection.

In another embodiment, the invention provides a method of preventing, diminishing or reducing the incidence of systemic fungal infection in a subject, said method comprising contacting a site of or predisposed to infection with:
a. a substrate
b. a gel or film comprising a polymer attached thereto, on at least a portion of a surface of said substrate; and
c. at least one effector compound associated with said gel or film;
   wherein said at least one effector compound is associated with the prevention, diminiution or reduction of the incidence of said fungal infection.

In another embodiment, the invention provides a method of preventing, diminishing or reducing the incidence of systemic fungal infection in a subject, said method comprising contacting a site of or predisposed to infection with:
a. a substrate
b. a foam or particle comprising a polymer attached thereto, on at least a portion of a surface of said substrate; and
c. at least one effector compound associated with said foam or particle;
   wherein said at least one effector compound is associated with the prevention, diminiution or reduction of the incidence of said fungal infection.

In one embodiment, the methods of this invention, provide for treatment, prevention, mitigation, reduction of incidence, reduction of severity, reduction of pathogenesis of infection, which is inclusive of local, or in other embodiments, systemic infection.

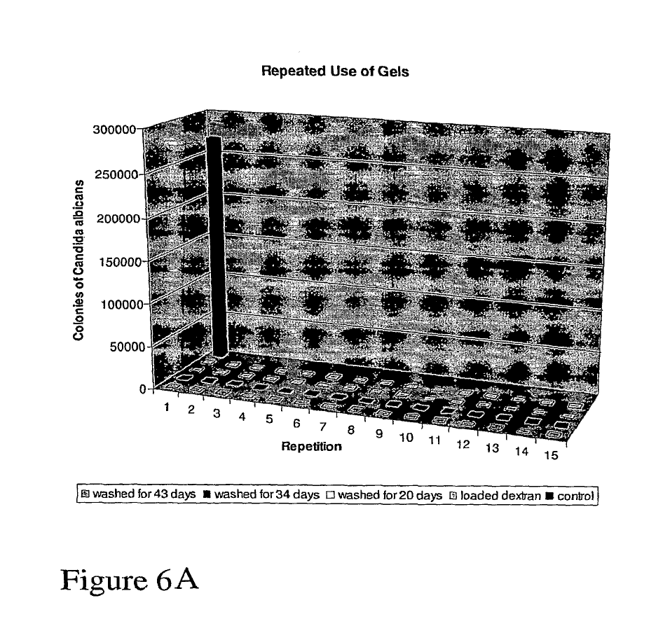

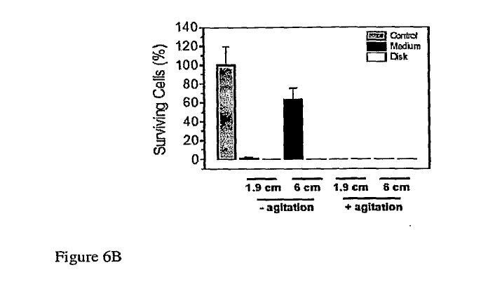

In one embodiment, the methods of this invention, provide for effects on infection and biofilm formation for extended periods of time. Such prolonged activity was exemplified herein in FIG. 6, wherein Amphotericin B-modified polymer disks and vehicle controls were repeatedly exposed to fresh Candida cultures and colony counts were obtained from the supernatants of control, but not Amphotericin B-modified polymer disks. Disks loaded with Amphotericin B (non-covalent association) were as active as their covalently attached counterparts, in terms of their fungicidal potency.

In one embodiment, the term “loaded” refers to any type of association of the compound with the foams, films, gels, compositions, or any combination thereof, of this invention. In one embodiment, “loaded” refers to the existence of any type of interaction between the compound and the foams, films, gels, compositions, or any combination thereof, of this invention. In one embodiment, the term “loaded” refers to the existence of a hydrogen bond, or in another embodiment, a covalent bond, or in another embodiment, van der Walls interaction, or in another embodiment, π-π interactions between the compound and the foams, films, gels, compositions, or any combination thereof, of this invention. In one embodiment, the term “loaded” refers to any means of association, interaction, bonding or attachment of a compound to or with the foams, films, gels, compositions, or any combination thereof, of this invention, as will be appreciated by one skilled in the art.

In one embodiment, the effects on infection and biofilm formation of the coated materials of this invention, are reflected in their persistence over a course of time, for example, for materials packaged and maintained under typical conditions, whereby several months to years after packaging does not significantly diminish such activity. In another embodiment, the effects of the coated materials of this invention, reflected in their persistence over a course of time, are for example, for materials which are subjected to repeat washings, or repeat contact with body fluids over a course of time, in situ, for example, suture material, scaffolding material, any type of implant, prosthesis, etc. where activity is maintained despite exposure to for example, aqueous environments. Similarly, and in some embodiments, the effects of the coated materials of this invention, reflected in their persistence over a course of time, are for example, for materials which are repeatedly subjected to harsh environmental conditions, such as materials exposed to sea water, for example a ship's hull, or in another embodiment, machinery involved in water purification, or water supplies, or in another embodiment, materials exposed to solvents, such as in the preparation of pharmaceuticals, or in another embodiment, materials subjected to repeat washings, for use in the food industry for preparations for mass consumption, or any other suitable application.

In one embodiment, the methods of this invention provide for diminished systemic toxicity of the effector compound, when the coated materials are applied to, implanted within, or contacted with a subject.

As exemplified herein, hydrogels which incorporated, for example, Amphotericin B, maintained the compound within the hydrogel, regardless of whether the effector compound was covalently bound to, or non-covalently associated with the gel (Example 1). Moreover, as exemplified herein, the modified polymer disk was non-toxic when implanted in a mammalian subject (FIG. 10). In one embodiment, such properties enable incorporation of smaller amounts of effector compound within a coated material or composition of this invention, due to the prolonged activity of the compound, or in another embodiment, slow release kinetics of the compound, etc., as will be appreciated by one skilled in the art. In one embodiment, such properties result in diminished systemic toxicity, via, in one example, delivery of smaller quantities of the effector compound, which may be toxic. In another embodiment, the diminished systemic toxicity is due to diminished release from the substrate, which in some embodiments is due to a lack of metabolization of the effector compound to one that has moderate to pronounced systemic toxic effects.

In another embodiment, this invention provides a method of preventing, diminishing or reducing the incidence of microbial attachment to a biomedical coated material, the method comprising attaching to a portion of a surface of said coated material, a gel or film comprising a polymer dispersed in a solvent comprising at least one effector compound, wherein the at least one effector compound is associated with the prevention, diminishment or reduction in incidence of microbial attachment to the coated material.

In another embodiment, this invention provides a method of preventing, diminishing or reducing the incidence of microbial attachment to a biomedical coated material, the method comprising attaching to a portion of a surface of said coated material, a foam or particle comprising a polymer dispersed in a solvent comprising at least one effector compound, wherein the at least one effector compound is associated with the prevention, diminishment or reduction in incidence of microbial attachment to the coated material.

In some embodiments, gels, foams, films, particles, and/or compositions of this invention are provided at a site of wound in a subject, whereby the material comprises cytotoxic substances, which serve to destroy or diminish the amount of diseased cells or tissue at the wound site, and in some embodiments, concurrently comprise tissue-promoting materials to selectively promote formation of healthy tissue. For example, and in one embodiments, various antimicrobial compounds can be incoporated in infected bone tissue, which serves to kill the source of infection. The same gels, foams, films, particles, compositions of this invention, etc, according to this embodiment, may comprise stem cells and bone morphogenetic proteins, which in turn promote new bone formation, which may replace bone tissue damaged as a result of infection.

In one embodiment, the invention provides a method of controlled release of an effector compound in a subject, said method comprising administering to said subject:
a. a substrate
b. a gel or film comprising a polymer, on at least a portion of a surface of said substrate; and
c. at least one effector compound associated with said gel or film;
   whereby said effector compound is released slowly, as a function of time, from said gel or film.

In one embodiment, the invention provides a method of controlled release of an effector compound in a subject, said method comprising administering to said subject:
a. a substrate
b. a foam or particle comprising a polymer attached thereto, on at least a portion of a surface of said substrate; and
c. at least one effector compound associated with said foam or particle;
   whereby said effector compound is released slowly, as a function of time, from said foam or particle.

In one embodiment, the invention provides a topical composition for controlled delivery of a compound of interest, the composition comprising:
a. a particle
b. a gel or film comprising a polymer attached thereto, on at least a portion of a surface of the particle; and
c. at least one compound of interest associated with the gel or film.

In one embodiment, the invention provides a topical composition for controlled delivery of a compound of interest, the composition comprising:
a. a particle
b. a foam or particle comprising a polymer attached thereto, on at least a portion of a surface of the particle; and
c. at least one compound of interest associated with the foam or particle.

In another embodiment, this invention provides a method of topical controlled delivery of a compound of interest to a subject, said method comprising topically administering to said subject a composition comprising:
a. a particle
b. a gel, foam or film comprising a polymer attached thereto, on at least a portion of a surface of said particle; and
c. at least one compound of interest associated with said gel or film.

In another embodiment, the invention provides a method of controlled delivery of a compound of interest to a subject, said method comprising administering to said subject a composition comprising:
a. a particle
b. a gel, foam or film comprising a polymer attached thereto, on at least a portion of a surface of said particle; and
c. at least one compound of interest associated with said gel or film.

In another embodiment, the invention provides a method of treating, preventing, diminishing incidence, prolonging remission, prolonging latency, preventing relapse, preventing latency, ameliorating symptoms, or a combination thereof, of a disease in a subject, said method comprising administering to said subject:
a. a substrate
b. a gel, foam or film comprising a polymer attached thereto, on at least a portion of a surface of said substrate; and
c. an effector compound associated with said gel or film;
   whereby said effector compound is associated with treating, diminishing incidence, prolonging remission, preventing relapse, ameliorating symptoms of a disease in said subject.

In another embodiment, the invention provides a method of treating, preventing, diminishing incidence, prolonging remission, prolonging latency, preventing relapse, preventing latency, ameliorating symptoms, or a combination thereof; of a disease in a subject, said method comprising administering to said subject:
a. a particle
b. a gel, foam or film comprising a polymer attached thereto, on at least a portion of a surface of said particle; and
c. a compound of interest associated with said gel or film;
   whereby said compound of interest is associated with treating, diminishing incidence, prolonging remission, preventing relapse, ameliorating symptoms of a disease in said subject.

In another embodiment, this invention provides a method for inhibiting, mitigating or delaying uptake of a compound in a subject, the method comprising administering to said subject:
a. a substrate
b. a gel, foam or comprising a polymer attached thereto, on at least a portion of a surface of said substrate; and
c. an effector compound associated with said gel or film;
   whereby said effector compound is associated with inhibiting, mitigating or delaying uptake of a compound in said subject.

In another embodiment, this invention provides a method for inhibiting uptake of a compound in a subject, the method comprising administering to said subject:
a. a particle
a gel, foam or film comprising a polymer attached thereto, on at least a portion of a surface of said particle; and
c. a compound of interest associated with said gel or film;
   whereby said effector compound is associated with inhiting uptake of a compound in said subject.

In some embodiments, the effector compound according to this aspect of the invention is a monoamine oxidase inhibitor, a Selective Serotonin Reuptake Inhibitor (SSRI), a norepinephrine uptake 2 inhibitor, a -serine transport inhibitor, a 5-HT2C serotonin receptor inhibitor, HMG-CoA reductase inhibitors, and others as will be known to those skilled in the art. Such compounds inhibit uptake of materials, which in turn have a therapeutic effect. For example, the SSRI prevents serotonin reuptake, which, in turn may be beneficial in certain neurological conditions.

In some embodiments, the effector compound is a receptor inhibitor, preventing uptake via the specific receptor, and/or preventing signal transduction through such a receptor, thereby exerting a therapeutic effect. For example, hormone receptor antagonists which prevent, for example, steroid hormone binding to the receptor, and signal transduction, in a subject with a hormone-dependent cancer. According to this aspect of the invention, and in one embodiment, prevention of proper signal transduction through the hormone receptor, in turn, prevents, mitigates, etc., cancer progression in the subject.

In another embodiment, the inhibitor inhibits viral uptake by a cognate receptor, thereby mitigating, abrogating, etc, infection, for example, in the case of HIV/AIDS.

In another embodiment, this invention provides a method for promoting, initiating or enhancing uptake of a compound in a subject, the method comprising administering to said subject:
a. a substrate
b. a gel, foam or film comprising a polymer attached thereto, on at least a portion of a surface of said substrate; and
c. an effector compound associated with said gel or film;
   whereby said effector compound is associated with promoting, initiating or enhancing uptake of a compound in said subject.

In another embodiment, this invention provides a method for inhibiting uptake of a compound in a subject, the method comprising administering to said subject:
a. a particle
b. a gel, foam or film comprising a polymer attached thereto, on at least a portion of a surface of said particle; and
c. a compound of interest associated with said gel or film;
   whereby said effector compound is associated with inhiting uptake of a compound in said subject.

In one embodiment, the invention provides a method of retaining an effector compound in a subject in active form, said method comprising administering to or implanting in said subject:

a. a substrate
b. a gel or film comprising a polymer attached on at least a portion of a surface of said substrate; and
c. at least one effector compound associated with said gel or film;
whereby said effector compound is associated with said gel or film and retains activity for a prolonged period of time. In some embodiments said prolonged period of time is at least 10, 20, 30, 40, or 50 days. In some embodiments said prolonged period of time is up to 20, 30, 40, 50, 60, 70, 80, 90, or 100 days; in some embodiments the compound, or a surface having said compound associated therewith, retains at least 25%, at least 50%, or at least 75% of its activity at the end of said time period. In some embodiments said activity is killing a pathogenic microorganism, which in some embodiments is a fungus.

The following examples are presented in order to more fully illustrate the preferred embodiments of the invention. They should in no way be construed, however, as limiting the broad scope of the invention

### EXAMPLES

### Materials and Methods

### Dextran Hydrogel Preparation:

Dextran acrylate (dexT70-VA) with a degree of substitution of 23% was synthesized as described previously (Ferreira et al., Biomaterials 2002, 23, 3957-3967). Briefly, dextran (10 g; from Leuconostoc mesenteroides, Mn=39,940, Mw=70,000; Fluka Chemie AG) and vinyl acrylate (1.21 g; Aldrich) were dissolved in DMSO (150 mL) and the reaction initiated by adding 1.5 g of Proleather (enzyme from Bacillus sp.; Amano Enzymes). The reaction mixture was shaken at 50° C. (250 rpm) for 72 h, and then precipitated in acetone. The precipitate was dissolved in water and dialyzed for 5 days against milli-Q water, at 4° C., and finally lyophilized. The products were characterized by ¹H NMR to assess the DS.

Dext70-VA gels (10 mm diameter and 1 mm thickness, before swelling) were obtained by photopolymerization reaction of aqueous solutions of dextVA. DextVA (200 or 400 mg) was dissolved in 1.8 mL of 0.2 M phosphate buffer pH 7.2 (or sodium citrate buffer pH 5.0) after which an ultraviolet photoinitiator, 2-hydroxy-1-[4-(hydroxyethoxy)phenyl]-2-methyl-1-propanone (Irgacure 2959, 0.5% w/v, 200 μL) was added. The polymerization reaction was initiated by ultraviolet light (ca. 4 mW/cm²), between two glasses with a 1 mm spacer, for 10 minutes. The gel was subsequently removed from the glasses, punched using stainless steel bars to yield cylinders with 10 mm diameter, and those cylinders immersed in ca. 5 mL of phosphate buffer pH 7.4 for 3-4 days, changing the buffer daily, at 2.5° C. For dextran-based hydrogels containing AcrPEGNHS (3400 Da or 5000 Da; Nektar), 10 or 2.0% (w/w) of AcrPEGNHS was mixed to the dextran solution before adding the photoinitiator. The polymerization reaction was performed as previously.

In addition, dextran hydrogels loaded with Amphotericin B were prepared as follows: To prepare dextran-based hydrogels containing amphotericin B, dextVA (400 mg) was dissolved in 1.8 mL of 0.2 M sodium acetate buffer pH 5.0 containing different percentages of AcrPEGNHS and 200 μL of photoinitiator was added in the last step. Buffer pH 5.0 was selected to decrease the hydrolysis rate of terminal NHS from AcrPEGNHS. After the polymerization reaction, the gel was punched using stainless steel bats, and 6 cylinders with 10 mm diameter were immersed in a solution of DMF: triethylamine (16 mL; 15:1) containing 20 mg of amphotericin B and 5 mg of 4-DMAP. The coupling reaction and loading was performed overnight, afterwich the gels were extensively washed with DMF (2-3 days) and then with phosphate buffered saline, pH 7.2 (for at least 3 days).

In addition, dextran hydrogels without PEG, loaded with Amphotericin B were prepared as follows:

6 disks were immersed in 16 mL of a solution of DMF:triethylamine (15:1) containing 20 mg of AmB (Sigma-Aldrich) and 5 mg of 4-dimethylaminopyridine. The loading was performed for 12 h, then the gels were washed with DMF (3 days) followed by PBS, pH 72 (3 days).

Preparation of Polyurethane and/or poly{lactic-co-glycolic) Acid (PLGA) Disks Loaded with Amphotericin B

Polymeric disks comprising polyurethane and/or poly{lactic-co-glycolic) acid (PLGA) loaded with Amphotericin B were prepared. One gram of polymers, in whatever proportion was desired (e.g. 50% PLGA-50% urethane) was dissolved in 10 ml chloroform. One ml of a 50 mg/ml solution of amphotericin B in DMSO was added and mixed thoroughly. Two hundred microliters of drug plus polymer solution were aliquoted into circular wells (approximately 2 cm in diameter) so that the solutions spread out evenly at the bottom, and were allowed to dry. The aliquoting was repeated four more times so that a total of 1 mL of drug plus polymer solution had been placed in each well. The solutions were allowed to evaporate in a chemical hood for two days in the dark. The discs were removed from the well, lyophilized overnight and stored until use.

### Preparation of Inulin and Poly(Ethylene Glycol) Hydrogels

Inulin acrylate with a degree of substitution of 28.7% was synthesized as reported previously (Ferreira, L., et al. (2002) Biomacromolecules 3, 333-41). Inulin hydrogels were obtained by the photopolymerization reaction of aqueous solutions of inulin acrylate (600 mg, in 0.2 M PBS) containing 0.056% (w/v) Irgacure 2959 Poly(ethylene glycol) gels were prepared by the photopolymerization of aqueous solutions of poly(ethylene glycol) diacrylate (5, 10 or 20% v/v, Mw of 700 Da, Sigma-Aldrich) in PBS containing 0.056% (w/v) of Irgacure 2959. In both cases, the polymerization reaction and gel loading with AmB was performed as described for dextran hydrogels.

### Gel Characterization

The swelling ratio at equilibrium (SR) was calculated according to the equation: SR=(Wₛ−W_{d})/W_{d}. To determine the swollen weight (Wₛ), swollen gels in PBS pH 74 were removed, blotted with filter paper to removesurface water and weighed. The gels were then lyophilized to determine the dry weight, W_{d}. Rheological experiments were carried out using the parallel plate geometry (8 mm diameter, steel) of an AR1000-N rheometer (TA Instruments). Equilibrium swelling conditions were maintained during rheological measurements by adding water on the lower plate of the geometry until the free lateral surface of hydrogels was completely wetted by the liquid. Hydrogels were subjected to stress sweep experiments (frequency of 1 Hz) to optimize the applied stress used in the frequency-oscillation experiments, recorded over a frequency range from 0.1 to 10 Hz

Gel loading efficiency was also determined. Amphogels were freeze-dried, then fragmented using a spatula and immersed in dimethylsulfoxide (DMSO, 500 μL) for 1 month. During this time, the gel suspension was washed three times with DMSO. The gel suspension was centrifuged at 6000 rpm, supernatant removed and replaced with fresh DMSO. This procedure was repeated 3 times during the 1 month period. The washing solutions (200 μL) were diluted with 200 μL of PBS pH 7.2 and analyzed by RP-HPLC (Atlantis dC-18, Waters) using 0.01 M Na₂H₂PO₄/CH₃CN (66/34 v/v), a flow of 0.5 mL/min, and a detection wavelength of 382 nm.

### Candida Growth Procedure:

Wildtype Candida albicans (CAN14) or Candida albicans strain SC5314 from frozen stocks were grown on YEP-agar plates. Yeast Peptone Dextrose (YPD) (50 mL) was inoculated with one colony of Candida overnight. The suspension was centrifuged (5 minutes, at 2000 rpm), the supernatant discarded, and cells were re-suspended in phosphate buffered saline (PBS) pH 7.2 (50 cc), then centrifuged (5 min. at 2000 rpm). This wash procedure was repeated once. The cells were counted on a haemacytometer and diluted to be at a concentration of 1×10⁷ of 4×10⁷ cells/ml with YNB medium with 50 mM glucose.

### Assessment of Candidal Viability on Disks (XTT Assay)

Polymer disks (1 cm diameter) were added into the wells of a 24-well plate, 1 ml of fetal bovine serum (PBS) was added and the disks were incubated at 37° C. for 12 hours, while shaking at 100 rpm. The FBS was removed and 1 ml of the standardized cell suspension was added. The disks were incubated for 2 hours at 37° C. while shaking at 100 rpm. Then each disk was carefully placed into a flesh 24-well plate containing 1 ml PBS 7.2 to remove non-adherent cells This procedure was repeated two times. The disks were then placed into 1 ml YNB media and incubated for 48 hours at 37° C. shaking at 100 rpm. Now the disks were washed again in two plates filled with PBS pH 7.2, whereupon they were placed into a 24-well plate containing 1 ml PBS pH 7.2. 50 μl of XTT (1 mg/ml in H₂O) and 4 μl of menadion (1 mM in acetone) were added. The disks were incubated for 5 hours at 37° C. 2 ml of PBS at pH 7.2 was added, the solution was transferred to a Falcon tube, and centrifuged for 5 min. at 2000 rpm. The OD of the supernatant was measured at 490 nm.

### Colony Counts

Disks were incubated for 2 h, unless specified otherwise, at 37° C. while shaking at 100 rpm. Then the disks were removed the remaining media were vigorously stirred then diluted 1:1000. 200 μL of the diluted media were plated on YEP agar plates. The disks were washed gently in 3×1 ml of fresh PBS to remove any non-adherent cells. Then the disks were crushed, vigorously stirred in 1 ml of PBS, and the suspension was diluted 1:1000. 200 μL of the diluted suspension was plated on YEP agar plates. The YEP plates were incubated at 37° C. for 24 h, and yeast colonies counted.

### Disk Biocompatibility

Male SV129 mice weighing 25 g (Charles River Laboratories, Wilmington, Mass.) were cared for in accordance with protocols approved by the US National Research Council. They were housed in groups, in 6 AM-6 PM light-dark cycles. Dextran-based hydrogels with or without AmB were sterilized by several washes in ethanol followed by washes in sterile PBS prior to implantation. Mice were anesthetized with isoflurane in oxygen, shaved and prepped in a sterile manner. An incision was produced in the dorsal midline, and subcutaneous pouches were extended down either flank by blunt dissection. One gel was deposited in each flank. At predetermined intervals, animals were euthanized with carbon dioxide, and the gels were removed along with adherent tissues and placed in 4% (v/v) neutral buffered formalin. After fixation for 24 h, the blocks were sectioned and stained (hematoxylin-eosin) using standard techniques, and analyzed by a blinded observer (DSK).

### In Vivo Fungicidal Activity

Animals were cared for in accordance with approved protocols of the US National Research Council. In each group, a total of five female BALB/c mice weighing 20-25 g were used. The mice were anesthetized with ketamine (100 mg/kg) and xylazine (5 mg/kg) intraperitoneally. Their backs were shaved, a midline incision was made in the skin above the mid-thoracic spine, and a pocket made subcutaneously by blunt dissection, extending 2-3 cm anteriorly. Individual Amphogels or dextran gels without AmB were inoculated with 1×10⁷ cells Candida albicans and incubated at 37° C. for 90 min, then placed in a subcutaneous pocket. The incision was closed with 3-0 vicryl sutures. Three days after inoculation, animals were sacrificed with carbon dioxide, and the disks were removed aseptically, weighed and used for enumeration of cells or microscopical examination. For the enumeration of cells the discs were homogenized, the suspension diluted and cultured in Sabouraud Dextrose Agar plates at 37° C. After 48 h, the number of colony forming units (cfu) were counted and expressed as cfu/g of disc.

### Statistical Analysis

The statistical significance of differences between groups was assessed by one-way analysis of variance, with Bonferroni correction as necessary for subsequent tests, using GraphPad Prism 4.0 (San Diego, Calif.). Results were considered significant when P □0.05.

### Example 1

### Hydrogels Incorporating AntiFungals are Fungicidal

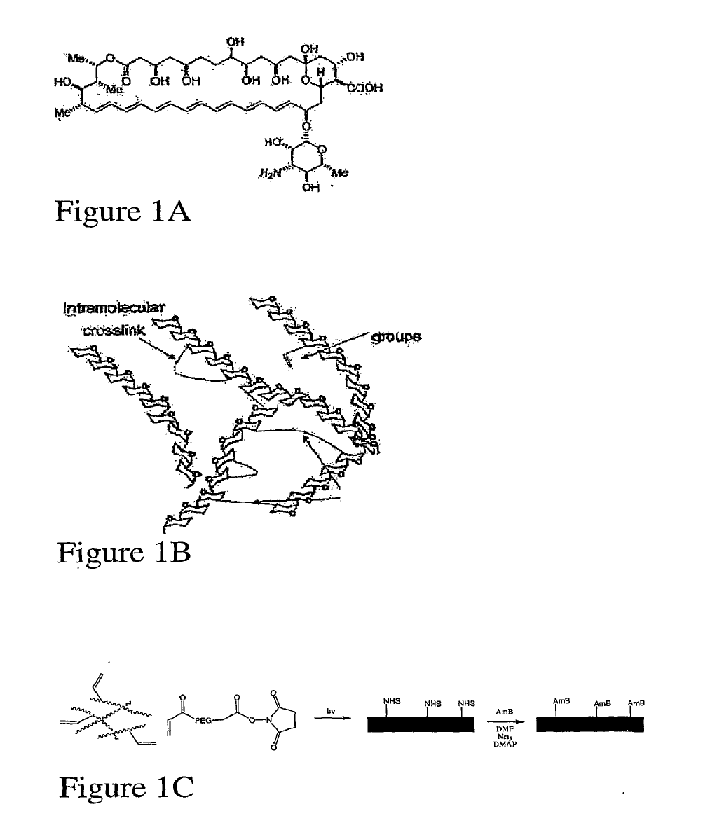

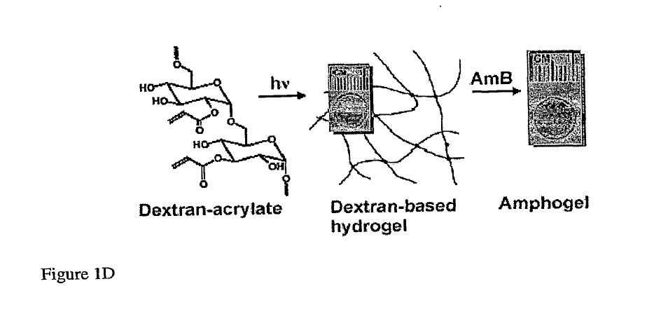

In order to determine whether Amphotericin B could successfully be incorporated within hydrogel matrices, dextran hydrogels were covalently bound to Amphotericin B, as described. A schematic of Amphotericin B is provided in FIG. 1A, and a schematic of an acrylate-bearing hydrogel synthesized as described is provided in FIG. 1B. A schematic of the reaction for the formation of one embodiment of the hydrogel comprising covalently bound Amphotericin-B is provided in FIG. 1C.

Dextran-based hydrogels containing AmB (referred to herein as “Amphogels”) were produced by placing cross-linked dextran disks in AmB-containing dimethylformamide (DMF) solutions overnight, followed by 6 days of washing (FIG. 1D). The gels had a constant swelling ratio for up to 33 days (FIG. 1E) indicating little degradation during that period. The elastic behaviour of Amphogels was evaluated by the rheologic determination of storage modulus (G′), which is a measure of the energy stored and recovered per cycle of oscillatory deformation. The G′ at equilibrium for these networks was 53.4±10.0 kPa, which is comparable to values reported for similar polysaccharide-based hydrogels, indicating a soft material. To assess the drug-loading capacity of the hydrogels, they were incubated in solutions containing increasing concentrations of AmB, and their AmB content was determined by extraction of the drug in dimethylsulfoxide (DMSO) with subsequent analysis via HPLC. The maximal loading capacity of the hydrogels was approximately 1.1 mg of AmB per disk. The disks had average dry and wet weights of 13.8±0.8 mg and 71.4±27 mg (n=7), respectively. As there was no benefit in cell killing from using higher AmB loadings, amphogels with 0.17±0.14 mg/gel of AmB were used for subsequent studies (made with a loading solution containing 1.3 mg/ml AmB).

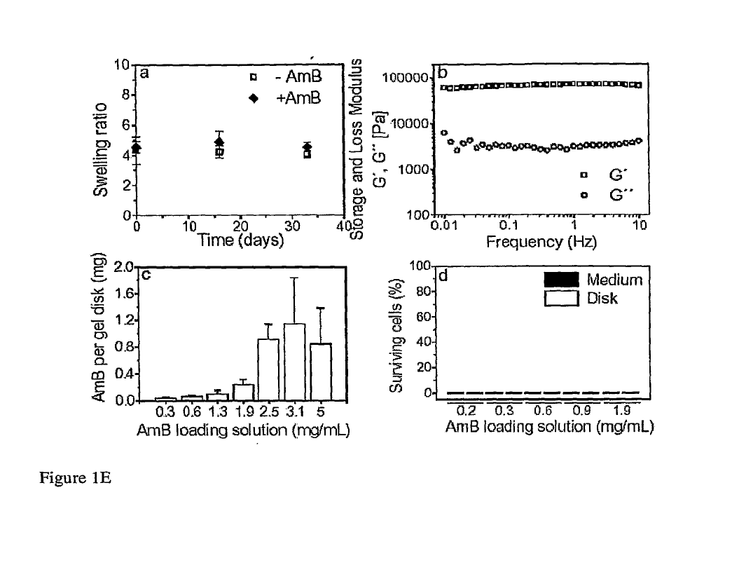

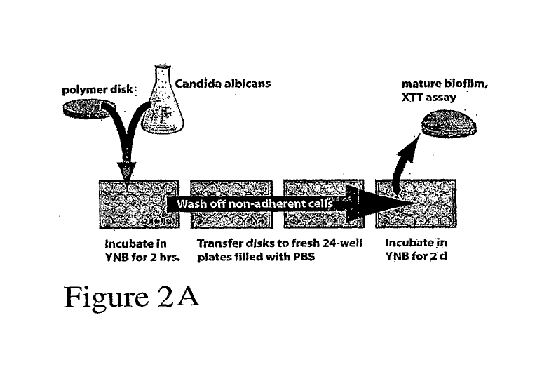

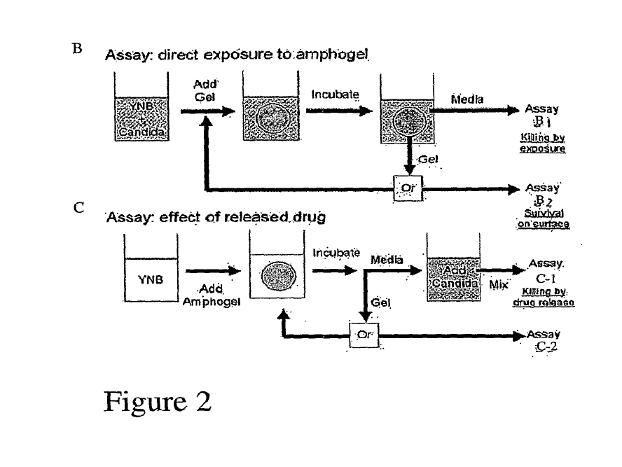

Polymer disks and Amphotericin B-modified polymer disks, were each incubated with Candida albicans as schematized in FIG. 2. The overall procedure is depicted in Panel A, with panels B and C outlining assay of effects of contact exposure versus that of released drug, respectively.

In order to determine the presence and extent of fungicidal effects of the Amphotericin B-modified polymer disks XTT assays were conducted to ascertain cellular viability (FIGS. 3A, 3B). Amphotericin B bearing surfaces lead to almost complete killing of the Candida.

The viability of yeast cells on the surfaces of amphogels or drug-free gels was assessed by incubating them in media containing C. albicans for 2 h, after which the disks were crushed and vigorously stirred. The suspension was plated on Yeast Extract Peptone Dextrose (YEP)-agar plates for 24 h and the number of yeast colonies was counted. Drug-free hydrogels did not inhibit fungal viability, whereas amphogels showed a marked reduction in fungal viability (FIG. 3C). Comparison of scanning electron micrographs (SEM) of the surface of hydrogels without AmB and amphogel (FIG. 3D) showed that the latter dramatically reduced the fungi on the surface.

Amphogels (n=4) placed in 1 ml Yeast Nitrogen Base (YNB) medium containing 1×10⁷ Candida albicans yielded no viable fungi in the medium (FIG. 3E), or on the surface of the Amphogel disks (data not shown). To assess the relative contributions from drug release and surface killing, Amphogels (n≧5) were placed into YNB medium and at predetermined time points the medium was collected. The viability of C. albicans in these media samples that had been exposed to Amphogel decreased significantly at 2 and 24 h (P<0.001) compared to cells grown in fresh YNB medium (FIG. 3F). After 24 h, the media no longer killed Candida. These studies indicated that there is some early release of fungicidal activity from the gel, which subsequently ceases. In contrast, the disks themselves maintained the capability to kill C. albicans for an extended period of time.

To further assess cell viability, colony counts were obtained for Candida cultured on Amphotericin B-containing and Amphotericin B-free surfaces (FIG. 4). No colony counts were obtained for cells cultured on surfaces containing Amphotericin B.

Scanning electron microscopy of the disks (FIG. 5) confirmed the formation of biofilms of the Candida on the untreated polymeric surfaces. No hyphae-formation was observed, however, on Amphogel-treated surfaces (5E, 5F).

In order to determine the longevity of effect of coated surface fungicidal activity, the control and modified disks were repeatedly exposed to fresh cultures and colony counts were obtained from the supernatants (FIG. 6A). Disks were exposed repeatedly up to 15 times, to C. albicans cultures, and colony counts from plated supernatants were obtained. In this case, hydrogels with Amphotericin B covalently attached to the matrix, or non-covalently associated with the matrix were included. Physically loaded dextran gels were as active as Amphogel, in terms of prolonged fungicidal activity, and repeat washing of the gels did not destroy their fungicidal potency.

Amphogel contact-driven fungicidal activity was also demonstrated by the following: Amphogels were incubated for 2 hours without mechanical agitation, either in 1 ml of media containing 1×10⁷ C. albicans in 1.9 cm diameter wells, or in 2 ml of media containing 2×10⁷ C. albicans in 6 cm diameter wells. While the cells in the 1.9 cm wells were completely killed, the majority of the cells in the 6 cm wells survived (approximately 65%; FIG. 6B). However, if the 6 cm wells were gently agitated during the 2 hours of incubation, there was no fungal cell survival in the media. Similar results were obtained when disks were pre-incubated in YNB for 5 days, which were therefore no longer releasing fungicidal activity. These findings are consistent with the possibility that direct contact with the Amphogel disk is necessary for fungal cell killing. In these experiments as well, no viable cells were recovered from the surfaces of the Amphogels.

Amphogels were amenable to storage by freeze-drying. Disks lyophilized then rehydrated in PBS for 24 h showed the same effectiveness in killing Candida as observed for Amphogels that were not freeze-dried (data not shown).

### Example 2

### Additional Examples of Hydrogels Incorporating an Anti-Fungal

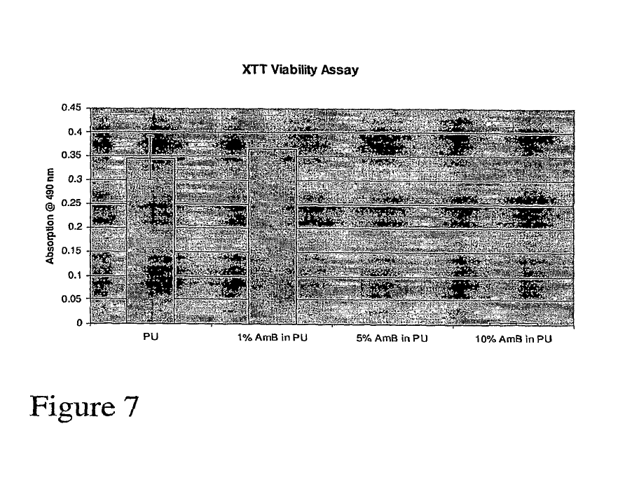

It was of interest to determine whether material other than dextran can successfully incorporate an antifungal, and effectively inhibit growth. Toward this end, polyurethane and/or poly{lactic-co-glycolic) acid (PLGA) loaded with Amphotericin B were prepared and evaluated for fungicidal activity (FIG. 7). Incorporation of Amphotericin B resulted in the eradication of Candida.

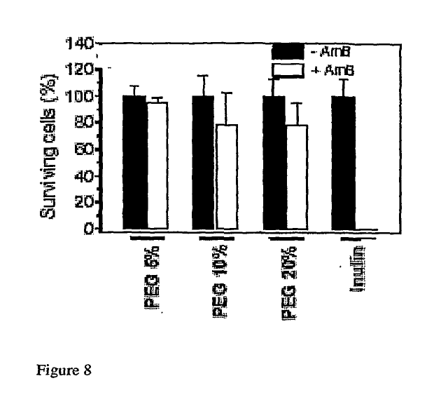

In addition, hydrogels comprised of additional sugars were prepared, with incorporated Amphotericin B. In this case inulin-hydrogels non-covalently incorporating Amphotericin B were prepared and retention of Amphotericin B was assessed and contrasted with polyethylene-glycol (PEG)-hydrogels non-covalently incorporating Amphotericin B and PEG-hydrogels without Amphotericin B. Hydrogels made from crosslinked PEG diacrylates did not retain AmB to visual inspection: the yellow color was lost after the first 24 h wash in DMF. PEG-based gels were demonstrably not able to retain Amphotericin B, as they were not active following just 2 washing-cycles, the sugar-based hydrogel retained fungicidal activity (FIG. 8), and was comparable to that observed for dextran-based hydrogels.

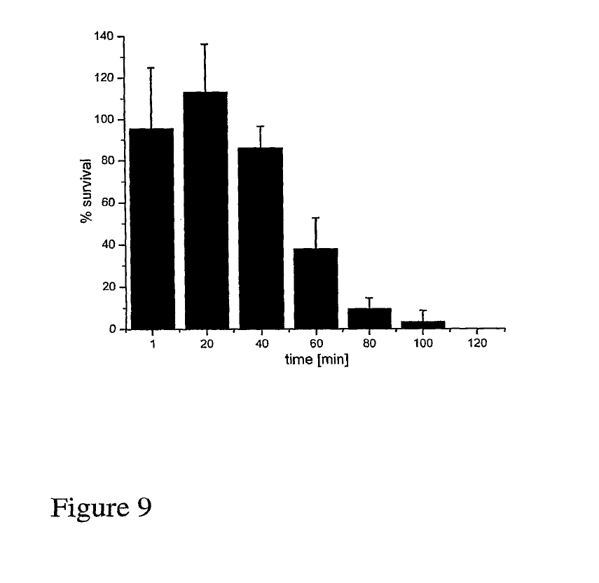

It was also of interest to determine the kinetics of the fungicidal activity. Towards this end, Amphogels were incubated with Candida albicans with shaking for varied amounts of time, and the percent survival was determined. At different time points, 200 μl of the suspension covering the discs were plated on YEP agar plates and incubated for 1 day at 37° C., at which point the colonies were counted and recorded. Each timepoint was taken from a different set of hydrogel discs (FIG. 9). As can be readily seen in the Figure, within 2 hours, contact with the Amphogel surface resulted in the complete killing of all cells.

### Example 3

### Implanted Hydrogels Incorporating Anti-Fungals are Biocompatible

In order to determine whether implanted hydrogels incorporating antifungals are biocompatible, hemocompatibility of amphogels was tested by exposing red blood cells to amphogels for 1 h and quantifying the release of free hemoglobin as a measure of cell lysis (FIG. 10A). No release was detected. The biocompatibility of amphogels and dextran gels without AmB in vivo was evaluated by subcutaneous implantation in mice. In all samples from both groups, there was minimal to mild inflammation at 3 days after implantation (FIG. 10B), and only mild to moderate inflammation at three weeks (FIG. 10C), both to gross inspection and by light microscopy. Tissue reaction was similar in both groups at both time points, and disks maintained their structural integrity.

The in vivo activity of an amphogel in killing C albicans was evaluated in a mouse model. Amphogels or hydrogels without AmB were inoculated with C albicans then implanted subcutaneously in mice for 3 days. Animals were then sacrificed and the disks were removed for enumeration of cells and microscopic examination. No fungal survival was observed with amphogels (log CFU per g of disk: 0±0, n=5), while the dextran hydrogel without AmB showed an average of 5.7±0.4 (n=5) log CFU per g of disk, i.e. almost 10⁶ CFU per g Of disk. SEM showed that amphogel surfaces did not have any Candida cells or biofilm attached (FIG. 10D). In some areas of the amphogels, a few host cells (mainly white blood cells) were observed (FIG. 10E). In contrast, dextran hydrogels without AmB were covered with Candida biofilm (FIG. 10F), Candida blastosphores, and white blood cells (FIG. 10G).

The foregoing has been a description of certain non-limiting preferred embodiments of the invention. Those of ordinary skill in the art will appreciate that various changes and modifications to this description may be made without departing from the spirit or scope of the present invention, as defined in the following claims.

1. A coated material comprising:
a. a substrate;
b. a gel or film comprising a polymer, said gel or film attached on at least a portion of a surface of said substrate; and
c. at least one effector compound associated with said gel or film.

2. The coated material of claim 1, wherein polymer is a block copolymer or a polysaccharide.

3.-4. (canceled)

5. The coated material of claim 4, wherein said polysaccharide is a poly(pyranose) or a poly(furanose) or a combination thereof, or a dextran or an inulin.

6. (canceled)

7. The coated material of claim 1, wherein said compound is covalently associated with said gel or film or forms a physical interaction with said gel or film.

8. (canceled)

9. The coated material of claim 1, wherein said substrate is a part of, or in the form of a bead, microparticle, bandage, suture, catheter, stent, valve, pacemaker, conduit, cannula, appliance, scaffold, central line, pessary, tube, drain, trochar or plug.

10. The coated material of claim 9, wherein said catheter is a PA, pericardial, pleural, urinary or intra-abdominal catheter.

11. The coated material of claim 9, wherein said drain is a cerebrospinal fluid drain.

12. The coated material of claim 9, wherein said tube is a tracheostomy, endotracheal or chest tube.

13. The coated material of claim 1, wherein said substrate is a part of, or in the form of an implant, a rod, a screw, an orthopedic appliance, a pipe lining, a reactor, or equipment which comes into contact with food or seawater.

14. (canceled)

15. The coated material of claim 1, wherein said compound is released slowly, over a course of time or is minimally released over a course of time.

16. (canceled)

17. The coated material of claim 1, wherein said coated material may be affixed, glued, sutured to the skin, pierce the skin, or serves as a portal through which other coated materials are passed through the skin.

18. (canceled)

19. The coated material of claim 1, wherein said effector compound is an antibiotic, an antiviral, an antifungal, an anti-helminth, an anti-inflammatory, an antihistamine, an immunomodulatory, an anticoagulant, a surfactant, a bronchodilator, an antibody, a beta-adrenergic receptor inhibitor, a calcium channel blocker, an ace inhibitor, a growth factor, a hormone, a DNA, an siRNA, a vector or any combination thereof.

20. The coated material of claim 19, wherein said antifungal is a polyene antifungal.

21. The coated material of claim 20, wherein said antifungal is Amphotericin B.

22. The process of claim 35 wherein the gel or film is covalently attached to the coated material and to the effector compound, said process further comprising the step of
chemically reacting said gel or film with the effector compound.

23. (canceled)

24. The process of claim 22, wherein reacting comprises activating said gel to produce an active ester, amine- or thiol-reactive group or photoreactive group in said gel or film.

25. The process of claim 24, wherein said ester is N-hydroxy-succinimide ester.

26.-34. (canceled)

35. A process for preparing a coated material comprising an effector compound associated thereto, said process comprising the steps of:
a. preparing a gel or film comprising a polymer dispersed in a solvent;
b. loading said gel or film with an effector compound; and
c. attaching said gel or film in (b) to at least a portion of a surface of a substrate;
thereby preparing a coated material.

36. The process of claim 35, wherein preparing said gel or film comprises the step of dispersing said polymer in water, dimethylsulfoxide, dimethylformamide or N-methylpyrrolidinone (NMP).

37. The process of claim 35, wherein attaching said gel or film to said substrate is via chemically reacting said gel or film with at least a portion of a surface of said substrate

38. The process of claim 37, wherein attaching comprises activating at least a portion of a surface of said coated material, and providing conditions whereby said gel or film reacts with said activated surface of said substrate.

39. The process of claim 35, wherein said polymer is a block copolymer or polysaccharide.

40. (canceled)

41. The process of claim 39, wherein said polysaccharide is a poly(pyranose) or a poly(furanose) or a combination thereof, or a dextran or an inulin.

42. (canceled)

41. The process of claim 35, wherein said effector compound is an antibiotic, an antiviral, an antifungal, an anti-helminth, an anti-inflammatory, an antihistamine, an immunomodulatory, an anticoagulant, a surfactant, a bronchodilator, an antibody, a beta-adrenergic receptor inhibitor, a calcium channel blocker, an ace inhibitor, a growth factor, a hormone, or any combination thereof.

44. The process of claim 43, wherein said antifungal is a polyene antifungal.

45. The process of claim 44, wherein said antifungal is Amphotericin B.

46. A method of preventing, diminishing or reducing the incidence of microbial attachment to a biomedical substrate or infection caused by introduction or implantation of a substrate in a subject, the method comprising attaching to a portion of a surface of said substrate, a gel or film comprising a polymer and at least one effector compound, wherein said effector compound is associated with the prevention, diminishment or reduction in microbial attachment or incidence of infection.

47. A method of preventing, diminishing or reducing the incidence of local fungal infection in a subject, said method comprising contacting a site of, or predisposed to infection with:
a. a substrate;
b. a gel or film comprising a polymer attached to said substrate in (a), on at least a portion of a surface of said substrate; and
c. said gel or film being associated with at least one effector compound;
wherein said at least one effector compound is associated with the prevention, diminiution or reduction of the incidence of said fungal infection.

48. The method of claim 47, wherein said compound is released slowly as a function of time, at said site of infection.

49. The method of claim 47, wherein said method results in diminished systemic toxicity of said effector compound.

50. The method of claim 47, wherein said effector compound is a polyene antifungal compound.

51. The method of claim 50, wherein said polyene antifungal compound is Amphotericin B.

52. The method of claim 47, wherein said substrate is a bead or particle ranging in size from about 50 nanometers-300 microns.

53. The method of claim 47, wherein said substrate is a contraceptive device.

54. (canceled)

55. A method of controlled release of an effector compound in a subject, said method comprising administering to or implanting in said subject:
a. a substrate;
b. a gel or film comprising a polymer attached on at least a portion of a surface of said substrate; and
c. at least one effector compound associated with said gel or film;
whereby said effector compound is released slowly, as a function of time, from said gel or film.

56. A topical composition for controlled delivery of a compound of interest, the composition comprising:
a. a particle;
b. a gel or film comprising a polymer attached on at least a portion of a surface of said particle; and
c. at least one compound of interest associated with said gel or film.

57. The method of claim 58 wherein the delivery is topical c.

58. A method of controlled delivery of a compound of interest to a subject, said method comprising administering to said subject a composition comprising:
a. a particle
b. a gel or film comprising a polymer attached on at least a portion of a surface of said particle; and
c. at least one compound of interest associated with said gel or film.

59. A method of treating, preventing, diminishing incidence, prolonging remission, prolonging latency, preventing relapse, preventing latency, ameliorating symptoms, or a combination thereof, of a disease in a subject, said method comprising administering to said subject:
a. a substrate
b. a gel or film comprising a polymer attached on at least a portion of a surface of said substrate; and
c. an effector compound associated with said gel or film;
whereby said effector compound is associated with treating, diminishing incidence, prolonging remission, preventing relapse, ameliorating symptoms of a disease in said subject.
